(19) United States
(12) Patent Application Publication
(10) Pub. No.: US 2016/0193385 A1
(43) Pub. Date: Jul. 7, 2016

(54) MESOSTRUCTURED POLYMER MEMBRANES AND OTHER ARTICLES

(71) Applicant: CHILDREN'S MEDICAL CENTER CORPORATION, Boston, MA (US)

(72) Inventors: Bozhi Tian, Chicago, IL (US); Daniel S. Kohane, Newton, MA (US)

(73) Assignee: Children's Medical Center Corporation, Boston, MA (US)

(21) Appl. No.: 14/911,792

(22) PCT Filed: Aug. 14, 2014
(86) PCT No.: PCT/US2014/050996

§ 371 Date: Feb. 12, 2016

Related U.S. Application Data
(60) Provisional application No. 61/866,661, filed on Aug. 16, 2013.

(51) Int. Cl. A61L 27/26; A61L 27/56; C08J 9/26; C08J 9/00; B01D 71/52; B05D 1/00; B01D 71/80; B01D 71/48; B01D 67/00; A61L 27/38; B05D 1/18

(52) U.S. Cl. CPC A61L 27/26; A61L 27/3826; A61L 27/56; C08J 9/26; C08J 9/0085; B05D 1/18; B05D 1/005; B01D 71/80; B01D 71/48; B01D 67/003; B01D 71/52; A61L 2300/64; C08J 2201/0464; C08J 2205/044; C08J 2207/10; C08J 2371/02; C08J 2467/04; B01D 2323/12; B01D 2325/02

(57) ABSTRACT

The present invention generally relates to porous membranes and other porous articles. In one aspect, the present invention is generally directed to porous membranes and other articles that have a pore size comparable to feature sizes of the extracellular matrix. Such articles may be useful, for example, for tissue engineering (e.g., as a substrate for culturing cells), as a filter, or for other applications. In some cases, the membranes may be formed from biocompatible and/or biodegradable materials. In some embodiments, such membranes may be formed using solvent evaporation induced self-assembly (EISA) techniques, although other techniques may be used in other embodiments. Still other aspects of the present invention are directed to methods of using such articles, kits involving such articles, and the like.

### RELATED APPLICATIONS

This application claims the benefit of U.S. Provisional Patent Application Ser. No. 61/866,661, filed Aug. 16, 2013, entitled “Mesostructured Polymer Membranes and Other Articles,” incorporated herein by reference in its entirety.

### GOVERNMENT FUNDING

Research leading to various aspects of the present invention was sponsored, at least in part, by the NIH, Grant No. GM073626. The U.S. Government has certain rights in the invention.

### FIELD

The present invention generally relates to porous membranes and other porous articles.

### BACKGROUND

Mesostructured constructs are important for a range of potential applications including tissue engineering, molecular detection, separation, environmental science, medicine, catalysis, and optics. For example, the extracellular matrix (ECM) has a quasi-ordered reticular mesostructure with feature sizes on the order of tenths to a few hundred nanometers. However, facile synthesis of mesostructured polymers with biomaterial compositions, or other properties, is needed, but is yet to be achieved.

### SUMMARY

The present invention generally relates to porous membranes and other porous articles. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments, the composition comprises a porous article comprising an amphiphilic block copolymer and a hydrophobic block copolymer. In some cases, the porous article comprises pores having an average pore size of between about 100 nm and about 1 micrometer, as determined using SEM.

The composition, in another set of embodiments, includes a porous article comprising an amphiphilic block copolymer and a hydrophobic block copolymer. In certain cases, the porous article has an average pore size of between about 100 nm and about 1 micrometer, as determined using SEM. In some instances, the porous article further comprises voids having an average dimension of between about 1 micrometer and about 100 micrometers, as determined using SEM.

In another aspect, the present invention is generally directed to a method. According to one set of embodiments, the method includes acts exposing at least a portion of a substrate to a solution comprising a solvent, where the solution comprises an amphiphilic block copolymer and a hydrophobic block copolymer; removing at least some of the solvent such that the amphiphilic block copolymer and the hydrophobic block copolymer form, on the substrate, a solid comprising the amphiphilic block copolymer and the hydrophobic block copolymer; and removing at least some of the amphiphilic block copolymer from the solid.

The method, in accordance with another set of embodiments, includes acts inserting, into spaces between a plurality of particles, a solution comprising a solvent, wherein an amphiphilic block copolymer and a hydrophobic block copolymer are each dissolved in the solvent, and wherein the particles have an average dimension of between about 1 micrometers and about 100 micrometers; and removing at least some of the solvent such that the amphiphilic block copolymer and the hydrophobic block copolymer form a solid comprising the amphiphilic block copolymer and the hydrophobic block copolymer.

In another aspect, the present invention encompasses methods of making one or more of the embodiments described herein, for example, porous membranes. In still another aspect, the present invention encompasses methods of using one or more of the embodiments described herein, for example, porous membranes.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1F illustrates the preparation and characterization of certain membranes in one set of embodiments;

FIGS. 2A-2E illustrate various mesostructured membranes, in another set of embodiments;

FIGS. 3A-3D illustrate shaping and patterning of certain polymer constructs, in still another set of embodiments; and

FIGS. 4A-4H illustrate certain mesostructured membranes used in certain biological applications, in accordance with yet another set of embodiments.

### DETAILED DESCRIPTION

The present invention generally relates to porous membranes and other porous articles. In one aspect, the present invention is generally directed to porous membranes and other articles that have a pore size comparable to feature sizes of the extracellular matrix. Such articles may be useful, for example, for tissue engineering (e.g., as a substrate for culturing cells), as a filter, or for other applications. In some cases, the membranes may be formed from biocompatible and/or biodegradable materials. In some embodiments, such membranes may be formed using solvent evaporation induced self-assembly (EISA) techniques, although other techniques may be used in other embodiments. Still other aspects of the present invention are directed to methods of using such articles, kits involving such articles, and the like.

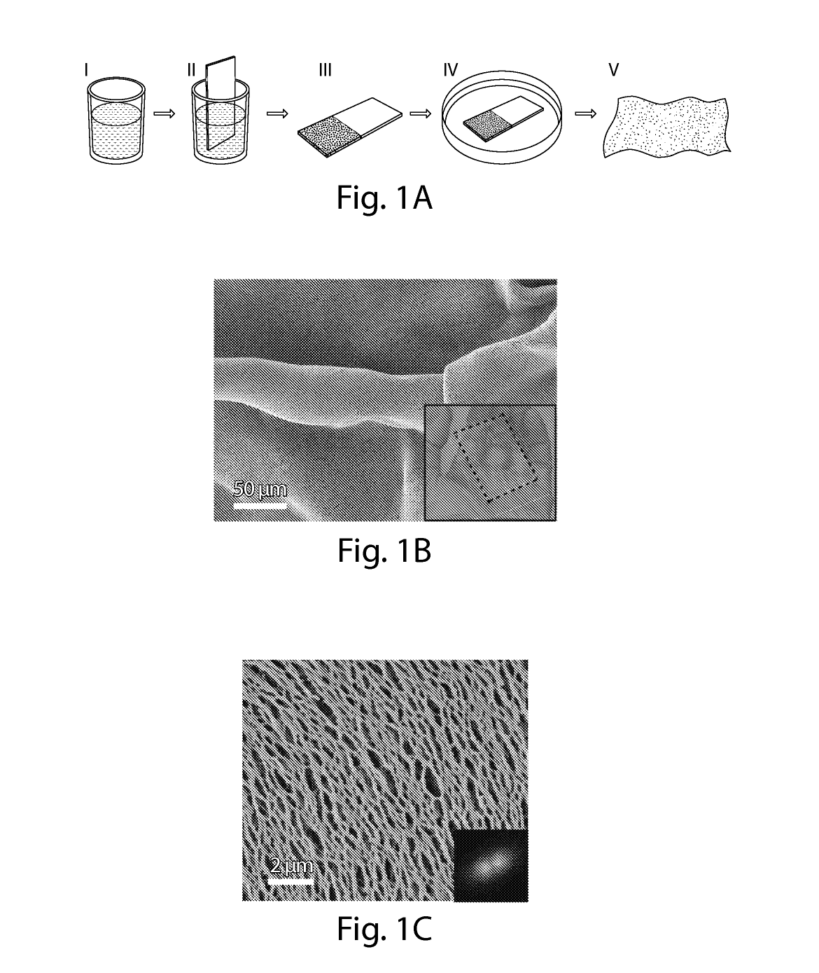

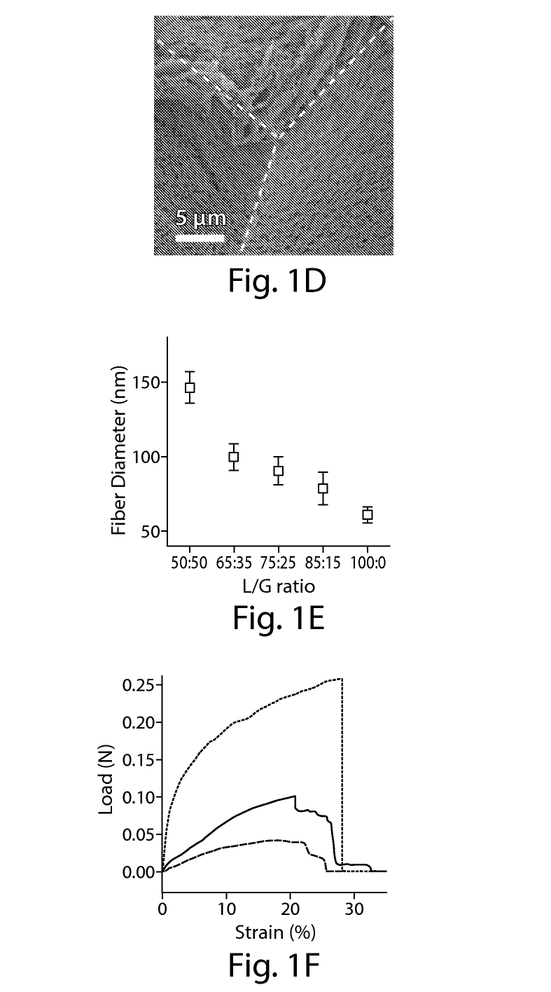

As mentioned, in one aspect, the present invention is generally directed to porous membranes or other porous articles. The pores within the membrane (or other article) may be of a size that is comparable to feature sizes of the extracellular matrix, which can be useful in promoting cell growth for certain applications. However, it should be understood that other pore sizes are also possible, and the present invention is not limited to only cellular or biological applications. In one set of embodiments, the pores have an average pore size of between about 100 nm and about 1 micrometer, or other dimensions as discussed herein. In addition, in some cases, the pores are not necessarily circular; for example, the pores may have an elongated appearance, such as those shown in FIG. 1C.

In one set of embodiments, the membrane (or other article) is formed from materials that are biocompatible and/or biodegradable. For example, the membrane may comprise amphiphilic polymers such as polyols, and/or hydrophobic polymers such as polyesters, which may be used to form the membrane, e.g., as discussed below. Non-limiting examples of polyesters include polylactic acid (PLA) and polyglycolic acid (PGA), and/or copolymers of these (i.e., poly(lactide-co-glycolide) acid or PLGA) and/or other polymers. Non-limiting examples of polyol include poly(ethylene glycol), poly(propylene glycol), and/or copolymers of these and/or other polymers. For example, in one embodiment, the polyol is a triblock poly(ethylene glycol)-poly(propylene glycol)-poly(ethylene glycol) copolymer.

In certain embodiments, an article such as a membrane can be formed by combining polymers (such as an amphiphilic polymer and a hydrophobic polymer, or other polymers as discussed herein) together in a solvent, coating at least a portion of a substrate with the solvent, and removing the solvent to form a polymeric article. The amphiphilic polymer within the polymeric article can also be at least partially removed, e.g., via leaching, to produce the final porous article. The polymeric article may be relatively thin in certain embodiments, e.g., such that the article can be used as a membrane. However, in other embodiments, the article may be thicker.

In one aspect, the pores within the article have an average pore size of between about 100 nm and about 1 micrometer. In other embodiments, the pores may have an average pore size that is at least about 50 nm, at least about 60 nm, at least about 70 nm, at least about 80 nm, at least about 90 nm, at least about 100 nm, at least about 125 nm, at least about 150 nm, at least about 175 nm, at least about 200 nm, at least about 225 nm, at least about 250 nm, at least about 275 nm, at least about 300 nm, at least about 350 nm, at least about 400 nm, at least about 450 nm, at least about 500 nm, at least about 600 nm, at least about 700 nm, at least about 800 nm, at least about 900 nm, or at least about 1000 nm. In some cases, the pores may have an average pore size of no more than about 1100 nm, no more than about 1000 nm, no more than about 900 nm, no more than about 800 nm, no more than about 700 nm, no more than about 600 nm, no more than about 500 nm, no more than about 450 nm, no more than about 400 nm, no more than about 350 nm, no more than about 300 nm, no more than about 250 nm, no more than about 200 nm, no more than about 175 nm, no more than about 150 nm, no more than about 125 nm, no more than about 100 nm, no more than about 90 nm, no more than about 80 nm, no more than about 70 nm, no more than about 60 nm, or no more than about 50 nm. Combinations of any of these dimensions are also possible in other embodiments. For example, in one embodiment, the pores in the article may have an average pore size of between about 100 nm and about 1000 nm, between about 125 nm and about 150 nm, between about 300 nm and about 350 nm, etc. If the pores are non-circular, e.g., as is shown in FIG. 1C, then the average pore size of a pore can be taken as the diameter of a circle having the same estimated area of the pore.

Any suitable technique can be used for determining average pore size. In some cases, the pore size may be determined by examining the material using visual or optical techniques, such as light microscopy or SEM (scanning electron microscopy), to estimate pore sizes. Other techniques, such as CT scanning or mercury intrusion porosimetry, may also be used to determine pore sizes in certain embodiments. In some cases, e.g., for articles having relatively homogenous pore distributions, several regions of an article can be randomly selected and analyzed to determine pore sizes in each region, then averaged together to determine the average pore size of the article.

In one set of embodiments, some or all of the pores may appear as elongated structures. For example, at least about 50%, at least about 60%, at least about 70%, at least about 80%, at least about 90%, or at least about 95% of the pores within an article may appear to be elongated, e.g., as determined visually or optically. In some cases, a pore may have an aspect ratio of at least about 1.5, at least about 2, at least about 2.5, at least about 3, at least about 3.5, at least about 4, at least about 4.5, at least about 5, at least about 6, at least about 8, at least about 10, etc. The aspect ratio of a pore may be taken as the ratio of its largest dimension compared to its smallest dimension. The dimensions may or may not necessarily be orthogonal to each other, for example, for pores that appear angled or curved. In some cases, the aspect ratio may be no more than about 15, no more than about 10, or no more than about 5. Combinations of these are also possible, e.g., the pores may have an aspect ratio greater than about 1.5 and less than about 10. In some cases, the aspect ratio can be determined or estimated visually; for example, an article may contain a plurality of pores, e.g., as is shown in FIG. 1C, and a random sampling of pores may be selected and their aspect ratios calculated to determine the average aspect ratio of the pores.

In certain embodiments, however, the article may not necessarily have a homogenous distribution of pores, and/or pore sizes or pore aspect ratios may not necessarily be uniformly distributed within the article. For example, the article may have a first region having a first average pore size and/or a first pore aspect ratio, and a second region having a second average pore size and/or a second pore aspect ratio different from those in the first region. The first and second average pore size and/or pore aspect ratio may each be any of the pore sizes or aspect ratios described herein. In some cases, there may be a relatively smooth gradient between the first region and the second region.

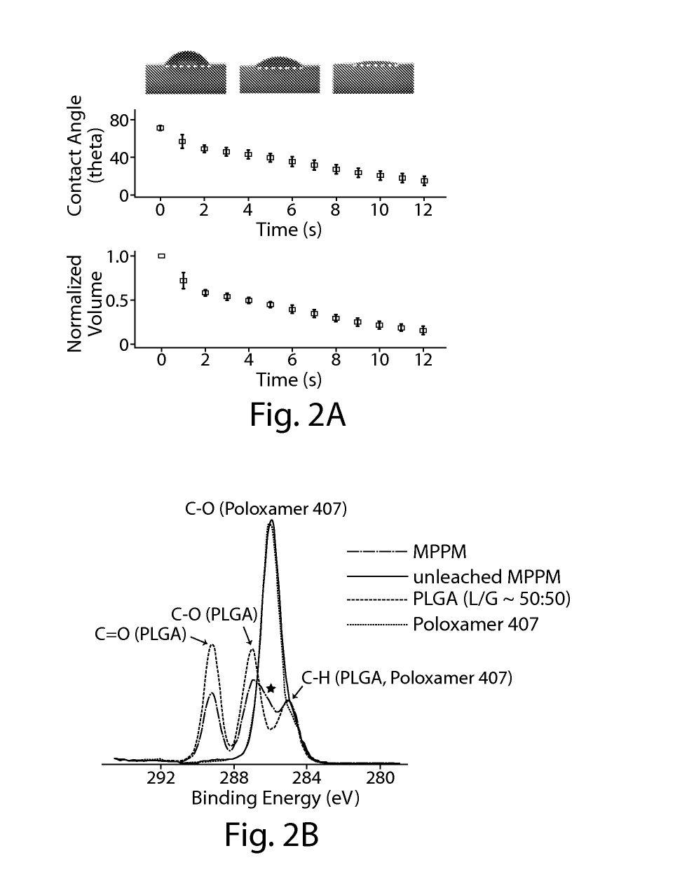

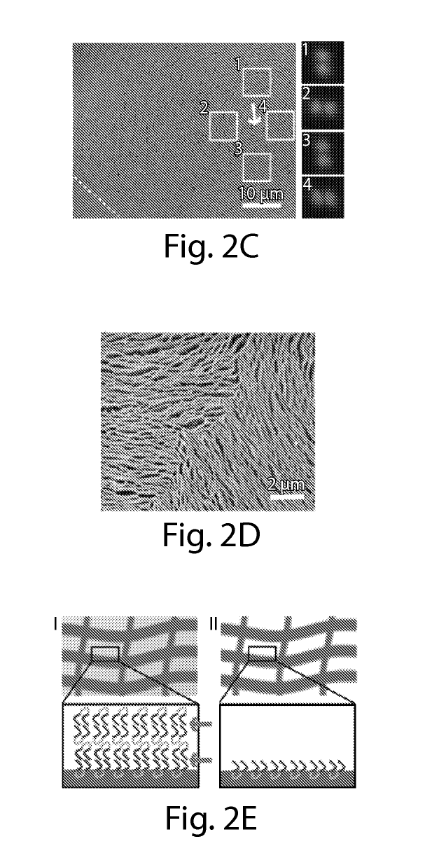

In some (but not all) aspects, the pores can be organized as pore domains within the article. The pore domains can appear visually as relatively concentrically-organized clusters of pores generally circularly arranged about a center region, e.g., as is shown in FIG. 1B or 2C. The pore domains can have a dimension of at least about 10 micrometers, at least about 15 micrometers, at least about 20 micrometers, at least about 30 micrometers, at least about 40 micrometers, at least about 50 micrometers, at least about 60 micrometers, at least about 70 micrometers, at least about 80 micrometers, at least about 90 micrometers, at least about 100 micrometers, at least about 110 micrometers, at least about 120 micrometers, at least about 130 micrometers, at least about 140 micrometers, at least about 150 micrometers, at least about 160 micrometers, at least about 170 micrometers, at least about 180 micrometers, at least about 190 micrometers, at least about 200 micrometers, at least about 300 micrometers, at least about 400 micrometers, or at least about 500 micrometers. The pore domains can also have a dimension of no more than about 500, no more than about 400, no more than about 300 micrometers, no more than about 200 micrometers, no more than about 190 micrometers, no more than about 180 micrometers, no more than about 170 micrometers, no more than about 160 micrometers, no more than about 150 micrometers, no more than about 140 micrometers, no more than about 130 micrometers, no more than about 120 micrometers, no more than about 100 micrometers, no more than about 90 micrometers, no more than about 80 micrometers, no more than about 70 micrometers, no more than about 60 micrometers, no more than about 50 micrometers, no more than about 40 micrometers, no more than about 30 micrometers, or no more than about 20 micrometers, and/or combinations of any of these (for example, between about 20 micrometers and about 200 micrometers). While it may be difficult to define exactly where a domain starts and stops to nanometer precision (e.g., due to the complex interface that can occur between 2 domains, as is shown in FIG. 2D), such pore domains can be readily estimated visually.

Without wishing to be bound by any theory, it is believed that such pore domains can occur during the formation process of the article, e.g., when polymers in a solvent nucleate onto a substrate to form the article. It is also believed that the deposition spreads outward from such nucleation sites, expanding until reaching other forming domains, thus resulting in the generally circular appearance for the pore domains. As it is expected that such nucleation regions occur essentially randomly, the pore domains are generally circular, but a given pore domain may be larger or smaller, or less circular, than other pore domains, based on the location of other nucleation sites randomly surrounding the pore domain.

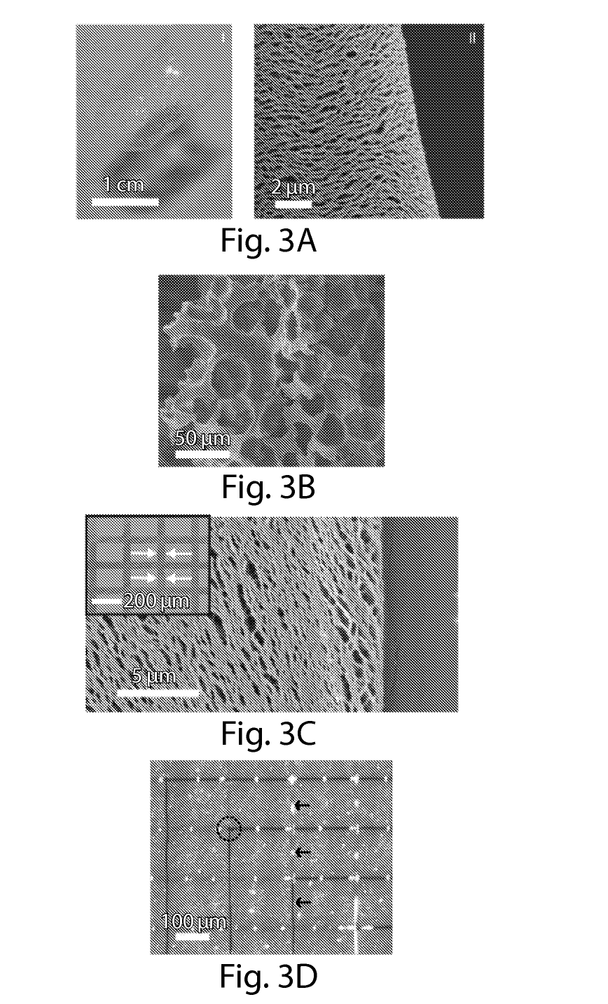

In some (but not all) cases, the article also comprises larger voids, e.g., having an average diameter of at least about 1 micrometer, and in some cases, at least about 2 micrometers, at least about 3 micrometers, at least about 4 micrometers, at least about 5 micrometers, at least about 6 micrometers, at least about 7 micrometers, at least about 8 micrometers, at least about 9 micrometers, at least about 10 micrometers, at least about 12 micrometers, at least about 15 micrometers, at least about 20 micrometers, at least about 25 micrometers, at least about 30 micrometers, at least about 40 micrometers, at least about 50 micrometers, at least about 60 micrometers, at least about 70 micrometers, at least about 80 micrometers, at least about 90 micrometers, or at least about 100 micrometers. In some cases, the voids may also have an average diameter of no more than about 100 micrometers, no more than about 80 micrometers, no more than about 60 micrometers, no more than about 40 micrometers, no more than about 20 micrometers, no more than about 10 micrometers, or no more than about 5 micrometers. Such voids can be readily identified visually or optically using techniques such as SEM, and are usually substantially larger than the pores. In addition, pores can often be observed within the walls defining the void spaces. As discussed below, such voids may be created by incorporating particles during formation of the article, which can be later removed to create the voids. A non-limiting example of an article comprising voids (in addition to pores, which are substantially smaller than the voids) can be seen in FIG. 3B.

In some embodiments, the article is substantially nonionic, and/or is formed from nonionic polymers, e.g., having no net charge or ions. For instance, in one aspect, the article comprises an amphiphilic polymer and a hydrophobic polymer. Typically, a hydrophobic polymer is a polymer (or co-polymer) having a water contact angle (under ambient conditions) of at least about 45°, at least about 50°, at least about 60°, at least about 70°, at least about 80°, at least about 90°, etc. Examples of hydrophobic polymers include polyesters, polycaprolactone, polyorthoesters, polyglycerols, poly(sebacate acrylate)s, poly(glycerol-co-sebacate acrylate), or the like. An amphiphilic polymer is a polymer comprising at least a first repeat unit that is hydrophobic and a second repeat unit that is hydrophilic (or not hydrophobic). Examples include, but are not limited to, poly(ethylene glycol-co-propylene glycol). In one set of embodiments, the polymer is a copolymer, e.g., comprising at least two different types of repeat units. In some cases, the copolymer may be a block copolymer; for example, the article may comprise an amphiphilic block copolymer and/or hydrophobic block copolymer.

In one set of embodiments, the amphiphilic polymer includes a polyol and/or the hydrophobic copolymer includes a polyester. A polyester typically contains an ester functional group in its backbone structure. In some cases, the ester functional group may be part of its repeat unit. The polyester can be biodegradable and/or biocompatible in certain instances. In addition, in some cases, the polyester may also contain other repeat units, e.g., as in a copolymer. Examples of polyesters include, but are not limited to, polylactide or polylactic acid (PLA), polyglycolide or polyglycolic acid (PGA), polycaprolactone, polyorthoesters, polyhydroxybutyrate, or the like. In some embodiments, copolymers of any of these and/or other polymers may be used, e.g., poly(lactide-co-glycolide) acid.

The polyester (or other hydrophobic polymer) may have any suitable molecular weight. For example, the polyester (or other hydrophobic polymer) may have a molecular weight of at least about 10 kDa, at least about 15 kDa, at least about 20 kDa, at least about 25 kDa, at least about 30 kDa, at least about 40 kDa, at least about 50 kDa, at least about 60 kDa, at least about 70 kDa, at least about 80 kDa, at least about 90 kDa, at least about 100 kDa, at least about 125 kDa, at least about 150 kDa, at least about 175 kDa kDa, at least about 200 kDa, etc. In some cases, the molecular weight of the polyester may be no more than about 200 kDa, no more than about 175 kDa, no more than about 150 kDa, no more than about 125 kDa, no more than about 100 kDa, no more than about 75 kDa, no more than about 50 kDa, no more than about 25 kDa, etc., and/or combinations of any of these (for instance, between about 10 kDa and about 150 kDa).

If lactide and glycolide are present in a polyester, they may be present in any suitable ratio. For example, the mass ratio between lactide and glycolide can be at least about 1:100, at least about 1:50, at least about 1:30, at least about 1:20, at least about 1:10, at least about 1:7, at least about 1:6, at least about 1:5, at least about 1:3, at least about 1:2, at least about 1:1, at least about 2:1, at least about 3:1, at least about 5:1, at least about 6:1, at least about 7:1, at least about 10:1, at least about 20:1, at least about 30:1, at least about 50:1, at least about 100:1, or the like. In some embodiments, the mass ratio between lactide and glycolide may be less than about 100:1, at least about 50:1, at least about 30:1, at least about 20:1, less than about 10:1, less than about 7:1, less than about 6:1, less than about 5:1, less than about 3:1, less than about 2:1, less than about 1:1, less than about 1:2, less than about 1:3, less than about 1:5, less than about 1:6, less than about 1:7, less than about 1:10, less than about 1:20, less than about 1:30, less than about 1:50, less than about 1:100, or the like. Combinations of these are also possible, e.g., the mass ratio between lactide and glycolide may be between about 1:100 and about 100:1, between about 1:5 and about 5:1, between about 1:2 and about 2:1, or the like. In some cases, the mass ratio of lactide but not glycolide may be about 0:100, about 15:85, about 25:75, about 35:65, about 50:50, about 65:35, about 75:25, about 85:15, or about 100:0, or the mass ratio may be between any of these ratios (e.g., between about 0:100 and about 15:85, between about 0:100 and about 25:75, between about 35:65 and about 85:15, etc.).

A polyol is a polymer whose repeat units are connected by ether (—O—) bonds. For example, the polyol may include repeat units such as (—CH₂—O—), (—CH₂—CH₂—O—), (—CH₂—CH₂—CH₂—O—), (—CH₂—CH(CH₃)—O—), (—CH₂—CH₂—CH₂—CH₂—O—), or the like. In addition, in some cases, the polyol may also contain other repeat units, e.g., as in a copolymer. In some embodiments, the polyol may be chosen to be biodegradable and/or biocompatible. Non-limiting examples of polyols include poly(ethylene glycol), poly(propylene glycol), poly(tertramethylene ether) glycol, or the like. In some cases, more than one polyol may be used, e.g., as separate polymers, or combined together into a copolymer. For example, the copolymer may be a copolymer of two or more polyol repeat units, in any suitable proportion or ratio. In one set of embodiments, for example, the copolymer is a triblock copolymer of poly(ethylene glycol)-poly(propylene glycol)-poly(ethylene glycol), e.g., a poloxamer. The poloxamer may have about 50%, about 55%, about 60%, about 65%, about 70%, about 75%, about 80%, about 85%, about 90%, or about 95% poly(ethylene glycol) with the balance being poly(propylene glycol). The poloxamer may also have any suitable molecular weight, e.g., at least about 2100, at least about 2400, at least about 2700, at least about 3000, at least about 3300, at least about 3600, etc. Non-limiting examples of poloxamers include Poloxamer 407 or Pluronics F127 (about 3,600 molecular weight, about 70% poly(ethylene glycol)), Pluronics F108 (about 3,000 molecular weight, about 80% poly(ethylene glycol)), or Pluronics F98 (about 2,700 molecular weight, about 80% poly(ethylene glycol)), etc.

If an amphiphilic polymer (such as a polyol) and a hydrophobic polymer (such as a polyester) are each present within the article, e.g., as a blend or within the same copolymer, the amphiphilic polymer and the hydrophobic polymer can each be present in any suitable ratio within the article. In some embodiments, the ratio of hydrophobic polymer to amphiphilic polymer (e.g., polyester to polyol) in the article may be between about 1:1 and about 1:10, or between about 1:2 and about 1:8 by mass. In some cases, the mass ratio of polyester to polyol may be greater than about 1:1, greater than about 1:2, greater than about 1:3, greater than about 1:4, greater than about 1:5, greater than about 1:6, greater than about 1:7, or greater than about 1:8, and/or the mass ratio may be less than about 1:10, less than about 1:9, less than about 1:8, less than about 1:7, less than about 1:6, less than about 1:5, less than about 1:4, less than about 1:3, or less than about 1:2.

In addition, some embodiments, the article has a different weight ratio of hydrophobic polymer to amphiphilic polymer (e.g., polyester to polyol) within the center or bulk of the article, as compared to the surface of the article. For example, the center or bulk of the article may have a higher weight ratio of hydrophobic polymer to amphiphilic polymer than does the surface of the article. Additionally, in some cases, the article may comprise a first region with a first weight ratio of hydrophobic polymer to amphiphilic polymer (e.g., polyester to polyol) and a second region with a second weight ratio of hydrophobic polymer to amphiphilic polymer (e.g., polyester to polyol), where the first weight ratio is higher than the second weight ratio. The first and second weight ratios may be any of the ones described herein.

In one set of embodiments, at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% by weight of the article comprises the hydrophobic polymer and the amphiphilic polymer (e.g., the polyol and the polyester). In one set of embodiments, at least about 10%, at least about 15%, at least about 20%, at least about 25%, at least about 30%, at least about 35%, at least about 40%, at least about 45%, at least about 50%, at least about 55%, at least about 60%, at least about 65%, at least about 75%, at least about 80%, at least about 85%, or at least about 90% of the article, by weight, may be the polyol (or other amphiphilic polymer). In some cases, at least about 10%, at least about 15%, at least about 20%, at least about 25%, at least about 30%, at least about 35%, at least about 40%, at least about 45%, at least about 50%, at least about 55%, at least about 60%, at least about 65%, at least about 75%, at least about 80%, at least about 85%, or at least about 90% of the article, by weight, may be the polyester (or other hydrophobic polymer).

As mentioned, in some embodiments, the article may be formed out of a biocompatible or biodegradable polymer. For example, the hydrophobic polymer and/or the amphiphilic polymer may be biocompatible, and/or the hydrophobic polymer and/or the amphiphilic polymer may be biodegradable. It should understood that a biodegradable material may or may not also be biocompatible, and vice versa. A biodegradable material is one that is subject to degradation when exposed to physiological conditions (e.g., an aqueous environment at about 37° C. containing physiological salts at physiological concentrations, for instance, NaCl at 0.9% w/v at a pH of about 7.4). Typically, the degradation occurs on the time scale of weeks, months, or 1-10 years, i.e., when such degradation can readily be observed visually, e.g., as an alteration of the average pore shape or size, and/or as an alteration of the shape or size of the article as a whole, for instance, as observed visually. The degradation may occur through hydrolysis of one or more polymers within the article, or through other mechanisms such as enzymatic attack, phagocytosis, chemical reaction, or the like.

A biocompatible material is one that may be implanted into a subject, such as a human or other mammalian subject, without adverse consequences, for example, without substantial acute rejection of the material by the immune system, for instance, via a T-cell response, after at least a week after implantation. It will be recognized, of course, that “biocompatibility” is a relative term, and some degree of inflammatory and/or immune response is to be expected even for materials that are highly compatible with living tissue. However, non-biocompatible materials are typically those materials that are highly inflammatory and/or are acutely rejected by the immune system, i.e., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In some cases, even if the material is not removed, the immune response by the subject is of such a degree that the material ceases to function; for example, the inflammatory and/or the immune response of the subject may create a fibrous “capsule” surrounding the material that effectively isolates it from the rest of the subject's body.

In one aspect, at least some of the polymer within the article is present as fibers. One non-limiting example of such a fibrous structure can be seen in FIG. 1C, where the fibers have a stranded or “reticulated,” net-like appearance, with pores defined in the spaces between the fibers, i.e., the spacing between the fibers defines the average pore size. Such fibers may be readily observed visually or optically, e.g., using SEM or other suitable techniques. In some cases, the pores may have an elongated appearance, as created by the fibers. In certain embodiments, at least about 50%, at least about 55%, at least about 60%, at least about 65%, at least about 70%, at least about 75%, at least about 80%, at least about 85%, at least about 90%, at least about 95%, or about 100% by weight of the polymer within the particle is present as fibers.

In one set of embodiments, the fibers have an average diameter of at least about 20 nm, at least about 30 nm, at least about 40 nm, at least about 50 nm, at least about 60 nm, at least about 70 nm, at least about 80 nm, at least about 90 nm, at least about 100 nm, at least about 125 nm, at least about 150 nm, at least about 175 nm, at least about 200 nm, at least about 250 nm, at least about 300 nm, at least about 350 nm, at least about 400 nm, at least about 450 nm, or at least about 500 nm. In some cases, the fibers may have an average diameter of no more than about 500 nm, no more than about 450 nm, no more than about 400 nm, no more than about 350 nm, no more than about 250 nm, no more than about 200 nm, no more than about 175 nm, no more than about 150 nm, no more than about 125 nm, no more than about 100 nm, no more than about 90 nm, no more than about 80 nm, no more than about 70 nm, no more than about 60 nm, no more than about 50 nm, no more than about 40 nm, no more than about 30 nm, or no more than about 20 nm. In some instances, combinations of any of these are possible, e.g., the fibers may have an average diameter of between about 50 nm and about 500 nm, between about 100 nm and about 200 nm, etc. The average diameter can be estimated, e.g., visually or optically, using SEM or other suitable techniques.

In some aspects, the surface of the article, after formation, is relatively hydrophilic. For example, the article can have a contact angle of less than about 45°, less than about 40°, less than about 35°, less than about 30°, less than about 25°, less than about 20°, less than about 15°, less than about 10°, less than about 5°, etc. In addition, in some embodiments, the article may be relatively flexible or elastic. In some cases, the article may be folded without breaking or cracking the article. For instance, the article may have an average tensile modulus of at least about 0.5 MPa, at least about 1 MPa, at least about 2 MPa, at least about 3 MPa, at least about 5 MPa, at least about 10 MPa, at least about 20 MPa, at least about 30 MPa, at least about 50 MPa, or MPa, at least about 100 MPa, and/or the article may have an average tensile modulus of no more than about 100 MPa, no more than about 50 MPa, no more than about 30 MPa, no more than about 20 MPa, no more than about 10 MPa, no more than about 5 MPa, no more than about 3 MPa, no more than about 2 MPa, or no more than about 1 MPa. In one set of embodiments, the article is substantially free of silicates. For example, the article may contain less than 10%, less than 5%, less than 3%, or less than 1% silicate by weight.

Other materials may be present within the article as well, in accordance with certain aspects. For example, the article may include a material, such as a polymer, that is included within the polymer as the article is formed. In some embodiments, such materials may be used to alter or control the proprieties of the article. For example, such materials may be used to alter the hydrophobicity or hydrophilicity of the article, increase the biocompatibility or biodegradability of the article, or to alter the porosity of the article. The material may be present in any suitable amount, e.g., less than about 10%, less than about 9%, less than about 8%, less than about 7%, less than about 6%, less than about 5%, less than about 4%, less than about 3%, less than about 2%, or less than about 1% by weight. For instance, in one set of embodiments, a biological agent may be included within the article. Examples of biological agents include, but are not limited to, peptides or proteins, hormones, vitamins, lipids, carbohydrates, sugars, or the like. As a non-limiting example, the article may include one or more materials that promote cell adhesion, e.g., fibronectin, laminin, vitronectin, albumin, collagen, or peptides or proteins containing RGD (arginine-glycine-aspartate) sequences or cyclic RGD sequences. Many of these materials are commercially available.

The article can be formed as a membrane, in accordance with one set of embodiments. In some embodiments, the membrane may have sufficient structural integrity to be self-supporting, e.g., the membrane can be manipulated as a solid material, without requiring additional materials to prevent the membrane from falling apart, e.g., during use. In some cases, the membrane may have a thickness or smallest dimension of less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 7 micrometers, less than about 5 micrometers, less than about 4 micrometers, less than about 3 micrometers, less than about 2 micrometers, less than about 1 micrometer, less than about 0.5 micrometers, or less than about 0.3 micrometers. The membrane may be useful, for example, for various tissue engineering applications, as a filter, or the like. Examples of such uses include, but are not limited to, those described below.

In another set of embodiments, the article can be formed as a coating on a substrate. In some cases, the article may not necessarily be one that is self-supporting. For example, the article can be present as a coating on a substrate at a thickness or smallest dimension of less than about 100 micrometers, less than about 50 micrometers, less than about 30 micrometers, less than about 20 micrometers, less than about 10 micrometers, less than about 7 micrometers, less than about 5 micrometers, less than about 4 micrometers, less than about 3 micrometers, less than about 2 micrometers, or less than about 1 micrometer, etc. The coating can be present, for example, on a medical device, an implantable device, etc.

The article can also be formed as a solid material, in yet other embodiments. For example, the article may be formed as a solid structure suitable for implantation, as a reservoir to contain a drug (e.g., for drug delivery applications), or as a fabric (e.g., for textile applications, such as clothing, cloth, towels, wrappings, etc.), or other applications such as those described below. The article can also be formed as a tube, as a sheet, or any other suitable structure. In some cases, the solid may have a smallest dimension (e.g., a smallest cross-sectional dimension) of less than about 100 micrometers, less than about 50 micrometers, less than about 30 micrometers, less than about 20 micrometers, less than about 10 micrometers, less than about 7 micrometers, less than about 5 micrometers, less than about 4 micrometers, less than about 3 micrometers, less than about 2 micrometers, or less than about 1 micrometer, etc.

In some aspects, the article can be used in various tissue engineering applications. For example, the article can be formed from various biocompatible and/or biodegradable materials, such as those discussed herein. The article may be implanted into a subject, such as a human or a non-human subject, with cells or tissue cultured thereon, or without cultured cells in some embodiments (e.g., in applications where it is desired for the subject's own cells to enter the article). Examples of non-human subjects include monkeys or other (non-human) primates, dogs, cats, mice, rats, other mammals, or the like. As previously discussed, in some cases, the pores within the article are comparable to feature sizes of the extracellular matrix, and thus such articles may be useful to facilitate cell growth and/or to decrease rejection. For example, cells cultured on such article may exhibit behaviors similar to behaviors such cells would exhibit if cultured on extracellular matrix. In some embodiments, one or more cells may be cultured on the article, e.g., by plating one or more cells on the article and incubating them under suitable conditions to encourage cell culture and growth. For example, at least a portion of the article may be exposed to cell culture media, e.g., under suitable temperatures, humidities, gas concentrations, etc. Those of ordinary skill in the art will be familiar with techniques for culturing cells on a substrate. The cells may be mammalian cells, including human or non-human cells, and/or non-mammalian cells in some instances.

Any of a wide variety of tissue engineering articles are contemplated in various embodiments. The article may have any shape, e.g., a tube, a sheet, a membrane, a solid article, or the like, including those described herein. In one set of embodiments, the article is used as a skin graft or a corneal transplant. In another embodiment, the article may be formed into a tube (for example, one or more membranes may be formed and rolled into a tube, or a tube may be coated with an article, etc.), for use as a vascular replacement. In yet another set of embodiments, the article may be implanted into a subject, for example, as a tissue scaffold, and/or to promote wound healing. For instance, the article may be implanted into a subject without any cells cultured thereon, for example, such that cells from the subject can enter into the article (e.g., via the pores within the article). As previously discussed, the article may be biodegradable in some embodiments, and thus, the article may eventually degrade or dissolve, leaving the subject's own cells behind, e.g., in a suitable configuration. For example, the article may be used to replace cartilage in a subject.

As another example, the article may be applied to a wound, e.g., an internal and/or an external wound, and used to promote wound healing. In some cases, the article may contain growth factors, hormones, cytokines, etc. for promotion of wound healing. For instance, the article may be formed into a bandage, gauze, dressing, etc. that is applied to a wound on a subject, or the article may be internally implanted within a wound, e.g., to provide a scaffold to facilitate wound healing.

In some embodiments, cells are cultured on the article, e.g., before implantation into a subject, or for certain in vitro applications (for instance, for research, drug screening, or the like). The cells may be from the subject to which the article will be implanted into, or from a different subject. This may be useful, for example, to facilitate acceptance of the article within the subject, to facilitate the growth of certain cells within the article (for example, by application of suitable culture media, growth factors, hormones, cytokines, etc.), or the like.

In yet another set of embodiments, one or more cells may be encapsulated within the article. Without wishing to be bound by any theory, it is believed that the cells may be trapped within the article, e.g., if the pores are chosen to prevent or at least reduce the ability of cells to migrate through the article. In some cases, such articles can allow nutrients to flow to the cells and waste products to exit the article, while immunologically isolating the cells from the subject and preventing rejection or other adverse immune reactions from occurring. In some cases, the article may be formed with a relatively hollow or open center (or other spaces) suitable for containing cells. As a non-limiting example, pancreatic cells, such as islet cells, may be encapsulated within a porous article, to be transplanted into a subject (e.g., one with diabetes). As another example, neurons may be encapsulated within a porous article suitable for implantation into a subject, and used to produce serotonin, dopamine, or other suitable neurotransmitters or other compounds. As other examples, the cells may be corneal cells, skin cells, epithelium cells, adrenal cells, endothelium cells, etc.

In still another set of embodiments, one or more drugs or other suitable agents can be encapsulated within the article (e.g., in addition to and/or instead of one or more cells, such as those discussed herein). The drug may be, for example, physically contained within the article, surrounded by the article, chemically incorporated within the article (e.g., within a backbone structure of a polymer, such as a hydrophobic polymer and/or an amphiphilic polymer), etc. For example, the article may allow sustained- or controlled-release of a drug (or other agent) contained therein, over an extended period of time. Due to the porosity within the article, which may be controlled as discussed herein, the ability of a drug to exit the article may be hindered, thereby allowing slower release of the drug to occur, e.g., over an extended period of time. In addition, in some embodiments, the article may be formed from biocompatible materials and/or biodegradable materials. For example, the article may be formed from biodegradable materials such that, after implantation into a subject and subsequent delivery of drug (or other agent), the article need not be removed from the subject. The drug may be, for example, a growth factor (e.g., BMP, BDNF, EGF, erythropoietin, FGF, IGF, TGF-alpha, TGF-beta, TNF-alpha, VEGF, IL-1, IL-2, IL-3, IL-4, IL-5, IL-6, IL-7, etc.) or a steroid (e.g., a glucocorticoid such as dexamethasone, an anabolic steroid such as testosterone or nandrolone, a progestin, etc.)

Non-biological applications are also contemplated in other embodiments of the invention. For instance, in one set of embodiments, the article may be used as a wrapping, e.g., for food, gifts, consumer items, or the like. In another set of embodiments, the article may be used as a filter, e.g., of a fluid. For example, an article can be formed as a membrane (or other structure) and a fluid passed through the membrane, e.g., a liquid or a gas. Solids or larger species contained within the fluid (e.g., larger than the pore size of the membrane) may be at least partially retained from crossing the membrane, and can thus be trapped and prevented from crossing through (or the species may pass through, but at a reduced amount or rate). Accordingly, the fluid may be at least partially purified of such species. The article can have any of the pore sizes discussed herein, which may be useful, for example, for removing various species from the fluid. In addition, in some cases, the article may be formed from biodegradable materials, which may be useful, for example, for disposing of such membranes (e.g., in landfill) in an environmentally-friendly manner.

In another set of embodiments, the article may be formed into clothing or textile materials. As discussed herein, the articles may be relatively flexible or elastic in certain embodiments. The articles may thereby be formed into clothing or textiles, which may be “breathable” in some cases, e.g., due to the pores within the article, where sweat or moisture from a subject can evaporate through the pores within the article. In addition, in certain cases, the articles may be formed from biodegradable materials. For instance, the clothing or textiles may be designed to be “single-use” clothing (e.g., for applications such as surgical gowns, other medical clothing, towels for medical use, gauze, or the like), or the clothing or textiles may be designed to be disposed of in an environmentally-friendly manner.

In still another set of embodiments, the article may be used as a substrate for electronics, such as flexible electronics. Such articles may be relatively flexible, as discussed herein, which may be useful, for example, for creating flexible electronics, implantable electronics, disposable electronics, biodegradable electronics or the like. Electronic devices, including nanowires, may be positioned on the article. In some cases, as discussed herein, the article may also be formed from a biodegradable material, e.g., for disposal after use in an environmentally-friendly manner.

In addition, in one set of embodiments, the article may be formed into a material that is responsive to temperature. In certain embodiments, the article may have surface area change due to the shrinkage of polyesters under aqueous conditions and at different temperatures. In some cases, the article may include a polymer (e.g., poly(N-isopropylacrylamide), PNiPAMs) that becomes a liquid at relatively low temperatures, while forming a gel at relatively high temperatures, i.e., the polymer engages in reverse thermal gelation. Such behavior may also be reversible in certain embodiments, e.g., the article may be repeatedly gelled and/or liquefied by altering the temperature of the article. In certain cases, the area of the membrane at 50° C. is approximately half that at room temperature (about 25° C.).

Another aspect of the present invention is generally directed to systems and methods for making such membranes and other articles as discussed herein. For example, in one set of embodiments, the article is formed by coating a solvent containing polymer onto a substrate, and removing some or all of the solvent such that the polymer deposits or otherwise forms a solid on the substrate, e.g., as a coating or a membrane. In some cases, at least a portion of the polymer may be removed, e.g., to create pores.

For example, in one set of embodiments, a solvent may be chosen that one or more polymers used to form the article is at least partially soluble in. For instance, if the article includes a polyol and a polyester, a solvent may be chosen in which the polyol and the polyester are each soluble. For example, the solvent may be one that is hydrophobic and/or is not substantially miscible with water, e.g., the solvent visually stably forms a phase-separated layer when added to water and left undisturbed (even if some amounts of dissolution still can occur). Examples of suitable solvents include tetrahydrofuran (THF), acetone or ethyl acetate, or chlorinated solvents such as chloroform or dichloromethane. In certain embodiments, other materials may also be present within the solvent, for example, biological agents (e.g., peptides, proteins, etc.), or other materials desired to be within the final article. Other examples of suitable biological agent are discussed herein. In addition, in some embodiments, void-creating materials may be present within the solvent. For example, the void-creating materials may include particles that can later be removed to create voids within the article, as discussed below.

The solvent (containing polymer) may be placed on a substrate, such that the solvent can be removed, leaving behind a polymeric layer on the substrate. The substrate may be flat or planar, or non-planar in some embodiments. In one set of embodiments, the substrate is inert, e.g., such that the article can be removed from the substrate, e.g., as a single, self-supporting unit. For example, the substrate can be an inert material (e.g., a silicon material, a silicon oxide material, a rubber material, etc.). In another set of embodiments, the substrate may become part of the final article; for example, the substrate may be a medical device or an implant.

In some cases, at least a portion of the substrate is coated or treated, e.g., to alter the ability of the solvent or the polymer to coat the substrate. For example, the substrate may comprise a first region having a first affinity to the solvent and a second region having a second affinity to the solvent different from the first affinity. In some cases, the regions may be relatively small, e.g., to cause micropatterning by the polymer onto the substrate. For instance, a region may have a smallest dimension of less than about 100 micrometers, less than about 50 micrometers, less than about 30 micrometers, less than about 10 micrometers, less than about 5 micrometers, less than about 3 micrometers, less than about 1 micrometer, less than about 500 nm, etc. As an example, the substrate may be partially coated with a silane, such as (heptadecafluoro)-1,1,2,2-tetrahydrodecyldimethyl-chlorosilane, to alter the affinity of the surface to the solvent. Other examples of silanes include, but are not limited to, N-(2-aminoethyl)-3-aminoprophyltriethoxysilane, N-(2-aminoethyl)-11-aminoundecyltrimethoxysilane, N-cyclohexylaminopropyltrimethoxysilane, or 11-mercaptoundecyltrimethoxysilane. Those of ordinary skill in the art will be familiar with techniques for micropatterning a substrate.

The solvent may be coated or positioned on the substrate using any suitable technique. For example, the solvent may be dip-coated, spin-coated, sprayed, brushed, or dripped onto the substrate. The solvent may be coated on all, or only a portion, of the substrate. In some cases, the solvent is coated substantially uniformly on the substrate, although in other cases, the coating may be non-uniform.

After coating, some or all of the solvent may be removed, e.g., to cause coating or deposition of the polymer onto substrate. Any suitable technique may be used to dry or remove the solvent. For instance, the solvent may be exposed to a vacuum or reduced pressure environment (e.g., at a pressure less than ambient pressure), and/or the solvent may be exposed to an increased temperature, e.g., to speed up evaporation of the solvent. For example, the temperature may be at least about 0° C., at least about 10° C., at least about 20° C., at least about 30° C., at least about 35° C., at least about 40° C., at least about 45° C., at least about 50° C., at least about 55° C., at least about 60° C., etc. In another set of embodiments, evaporation of the solvent may occur merely by exposing the solvent to ambient temperature and pressure. In some cases, the environment surrounding the solvent may have an elevated relative humidity, e.g., to control the rate of drying or removal of the solvent. For instance, the relative humidity may be at least about 50%, at least about 60%, at least about 70%, at least about 80%, at least about 90%, or the relative humidity may be saturated.

In one set of embodiments, sufficient solvent is removed such that the polymer dissolved in the solvent forms, on at least a portion of the substrate, a solid article. In some cases, drying occurs to form a coating of at least about 1 micrometer, at least about 2 micrometers, at least about 3 micrometers, at least about 4 micrometers, at least about 5 micrometers, at least about 7 micrometers, at least about 10 micrometers, at least about 12 micrometers, at least about 15 micrometers, at least about 18 micrometers, or at least about 20 micrometers is formed. In certain embodiments, the coating is less than about 20 micrometers, less than about 18 micrometers, less than about 15 micrometers, less than about 12 micrometers, less than about 10 micrometers, less than about 8 micrometers, less than about 7 micrometers, less than about 6 micrometers, less than about 5 micrometers, less than about 4 micrometers, less than about 3 micrometers, less than about 2 micrometers, or less than about 1 micrometer thick. In some cases, the coating may have a thickness between any of these values, e.g., between about 3 micrometers and about 10 micrometers in thickness.

In some cases, pores may be created within the article via removal of at least a portion of the polymer. For example, if the article comprises a polyol (or other amphiphilic polymer), at least some of the polyol (or other amphiphilic polymer) may be removed from the article, thereby creating pores within the article. For instance, at least about 10%, at least about 20%, at least about 30%, at least about 40%, at least about 50%, at least about 60%, at least about 70%, at least about 80%, or at least about 90% of the polyol (or other amphiphilic polymer) by weight may be removed from the article, thereby creating pores within the article. The pores may have any of the shapes or configurations described herein.

Any suitable technique may be used to remove the polyol, or other amphiphilic polymer within the article, to form pores. For example, the polyol (or other amphiphilic polymer) may be exposed to a liquid or a solution that the polyol can at least partially dissolve in, or to a liquid or solution that can at least partially leach the polyol (or other amphiphilic polymer) from the article, e.g., physically and/or chemically. For instance, in one set of embodiments, the polymer may be exposed to an aqueous solution to at least partially remove the polyol (or other amphiphilic polymer). The aqueous solution may be pure water, or comprise water and one or more salts or other species dissolved or suspended therein.

In one set of embodiments, voids can be created within the article, e.g., using certain void-creating materials. The voids that are created may, in some cases, have an average diameter of at least about 1 micrometer, or any of the other dimensions discussed herein with respect to voids. As discussed, such voids can be readily identified visually or optically using techniques such as SEM. In one set of embodiments, the void-creating material is a material that is added during formation of the article, and is later removed, e.g., chemically or physically. Typically, the void-creating material is removed without substantially disturbing or disrupting the article or its porous structure. The void-creating material may be removed before or after pores are formed within the article, e.g., via removal of at least a portion of the article (for example, by exposure to a liquid or a solution that can leach polymer within the article).

As a non-limiting example, in one set of embodiments, the void-creating material comprises a particle comprising silica (SiO₂) and/or titanium dioxide (TiO₂), which can be at least partially removed from the article using suitable etchants such as HF (hydrofluoric acid) and/or HCl (hydrochloric acid) without disrupting the polymer components of the article. In some cases, at least about 50% by volume, at least about 75%, at least about 95%, or substantially the entire particle may comprise SiO₂, TiO₂, or a combination of SiO₂ and TiO₂.

In some cases, the silica or other void-creating material may be present as particles, e.g., having an average diameter of at least about 1 micrometer, and in some cases, at least about 2 micrometers, at least about 3 micrometers, at least about 4 micrometers, at least about 5 micrometers, at least about 6 micrometers, at least about 7 micrometers, at least about 8 micrometers, at least about 9 micrometers, at least about 10 micrometers, at least about 12 micrometers, at least about 15 micrometers, at least about 20 micrometers, at least about 25 micrometers, at least about 30 micrometers, at least about 40 micrometers, at least about 50 micrometers, at least about 60 micrometers, at least about 70 micrometers, at least about 80 micrometers, at least about 90 micrometers, or at least about 100 micrometers. In some cases, the particles may also have an average diameter of no more than about 100 micrometers, no more than about 80 micrometers, no more than about 60 micrometers, no more than about 40 micrometers, no more than about 20 micrometers, no more than about 10 micrometers, or no more than about 5 micrometers, which may be used to create voids having these dimensions once removed from the article. In some cases, the particles may be between any of these values; for example, the particles may have an average diameter of between about 50 micrometers and about 100 micrometers, between about 25 micrometers and about 60 micrometers, between about 10 micrometers and about 100 micrometers, etc.

Thus, for example, particles (or other void-creating materials) may be added to a solvent comprising polymers, and an article formed by removing the solvent, as previously discussed. After formation, pores may be created within the article via removal of at least a portion of the polymer. The particles (or other void-creating materials) may be removed, e.g., by etching using a suitable etchant, such as HF and/or HCl, to create voids within the article, before or after formation of the pores.

U.S. Provisional Patent Application Ser. No. 61/866,661, filed Aug. 16, 2013, entitled “Mesostructured Polymer Membranes and Other Articles,” is incorporated herein by reference in its entirety.

The following examples are intended to illustrate certain embodiments of the present invention, but do not exemplify the full scope of the invention.

### Example 1

This example shows a simple and general solvent evaporation-induced self-assembly (EISA) approach to preparing concentrically reticular mesostructured polyol-polyester membranes. The mesostructures were formed by a self-assembly process without covalent or electrostatic interactions, which yielded feature sizes matching those of ECM. The mesostructured materials were nonionic, hydrophilic, and water-permeable, and could be shaped into arbitrary geometries such as conformally-molded tubular sacs and micropatterned meshes. Importantly, the mesostructured polymers were biodegradable, and were used as ultrathin temporary substrates for engineering vascular tissue constructs.

Solvent evaporation induced self-assembly (EISA) is a versatile means of producing two dimensional (2D-) and three dimensional (3D-) mesostructured films, and typically involves templating from surfactants or block copolymers. EISA permits control of the final structure by adjusting chemical and processing parameters (e.g., initial sol composition, pH, aging time, partial vapor pressures, convection, temperature, etc.). Additionally, this technique does not require lithography or external fields, and cheap, large-scale processes such as dip-coating can be used. It is a powerful strategy for creating highly structured multifunctional materials and devices.

The ability of mesostructured biodegradable and biocompatible polymers to mimic the structure of the extracellular matrix (ECM) holds great promise in regenerative medicine. Given the advantages of using biomaterials that have been extensively evaluated, this example used mesostructured polyol-polyester membranes (MPPM) by EISA (FIG. 1A), by blending poly(lactide-co-glycolide) acid (PLGA) or polylactide (PLA) with triblock poly(ethylene glycol)-poly(propylene glycol)-poly(ethylene glycol) (Poloxamer 407) in a 1:3 to 1:5 mass ratio in tetrahydrofuran (THF) (FIG. 1A, I). The solution was transferred onto planar or nonplanar substrates by dip-coating (FIG. 1A, II), followed by solvent evaporation at ambient conditions (25° C., 30-70% relative humidity), and humidified incubation (5% CO₂, 95% O₂, 37° C.) overnight (FIG. 1A, III) for solidification. The excess poloxamer-rich phase was then removed by leaching in phosphate buffered saline solution (1×PBS) (FIG. 1A, IV). Finally, the membranes were isolated from the substrate and rinsed with deionized water three times, and dried in air (FIG. 1A, V). Unless otherwise noted, the membranes in these examples were prepared from PLGA with a L/G ratio of 50:50 (5050 DLG 7E) and Poloxamer 407.

Scanning electron microscopy (SEM) of a ˜2 micrometers thick membrane after final drying showed smooth surfaces (FIGS. 1B, 1C). The membrane was flexible and foldable, with a smallest bending radius of ˜5 micrometers (FIG. 1B). The membrane could be peeled from an original glass substrate in water, float at a water-air interface, and be transferred onto another substrate (FIG. 1B, inset). The membrane surface featured reticular structures with fiber diameters of ˜146+/−11 nm (mean+/−SD) (FIG. 1C) that were locally aligned, as indicated by the fast Fourier transform (FFT) of the SEM image (FIG. 1C, inset). The average inter-fiber cavity width, ˜310-350 nm, was comparable to the spacing between natural ECM nanofibers. The fiber diameter and cavity width were ˜30-50 times larger than the corresponding feature sizes of mesostructured silica created by Poloxamer 407-mediated EISA. This observation suggested that the formation of quasi-ordered PLGA mesostructures was different from conventional lyotropic or thermotropic self-assembly, where the feature size is on the order of the amphiphilic chain length.

FIG. 1B is a SEM image of a ˜2 micrometer membrane with surface wrinkles. The inset is a photograph of a membrane transferred onto a glass slide; the dashed lines mark the membrane boundary. FIG. 1C is a SEM image highlighting the mesoscale surface topography. The inset is the fast Fourier transform (FFT).

The membrane had a uniform fibrous structure spanning the entire thickness (FIG. 1D) demonstrating the 3D mesostructure. Nanofiber diameter was inversely related to the polyester lactide-to-glycolide (L/G) ratio (FIG. 1E); polylactide (i.e., L/G=100:0) yielded the minimum fiber diameter of ˜60 nm. Hydrated membranes with different thicknesses could be axially stretched with failure strains of 20-28% (FIG. 1F). The calculated tensile modulus of hydrated MPPMs was ˜10-50 MPa, comparable to that of commercial polyglycolic acid yarns (Biomedical Structures LLC, Rhode Island) and articular cartilage.

FIG. 1D is a SEM image of a broken membrane edge, showing the 3D mesostructure. Dashed lines mark the edges of a membrane corner. FIG. 1E shows the effect of L/G ratio on fiber diameter. Data are means+/−SD, n=20. FIG. 1F shows membrane tensile characteristics. The membranes were 1 cm wide, and 12 micrometers (upper), 4 micrometers (middle) or 2 micrometers (lower) thick.

### Example 2

This example shows contact angles (FIG. 2A) of water on MPPMs, decreased from ˜70° to below 20° in 12 s. The contact area between droplets and MPPMs did not change detectably during that period. Normalized droplet volume above the MPPMs (FIG. 2A, lower panel) also decreased over time; less than 10% of the droplet was left above their surface after 12 s. These data demonstrate that the MPPM was hydrophilic and water permeable. A comparison of the compositions of nanometers-thin surfaces of MPPM, MPPM prepared without the leaching step, pure PLGA membrane, and pure Poloxamer 407 by carbon 1s X-ray photoelectron spectroscopy (XPS) (FIG. 2B) showed that MPPM was not pure PLGA; a C—O characteristic of Poloxamer 407 was identified at ˜286.1 eV. Given the probing depth of XPS (<10 nm), the poloxamer ‘brush’ was no more than a monolayer, as a thicker layer would yield a spectrum similar to that of pure Poloxamer 407 or the unleached MPPM. These data suggest that the MPPM is a polyester nanofibrous network with a surface layer of Poloxamer 407.

Multiple domains were observed in the MPPMs, with domain lateral dimensions of ˜20-200 micrometers (FIG. 2C, left panel). The variability of domain size could be reduced by surface patterning and/or controlled thermal treatment. Most domains had a concentric pattern, as revealed by both SEM and FFTs of four regions around the domain center (FIG. 2C, right panels 1-4). When crossing the domain boundary (dashed line in FIG. 2C, and FIG. 2D), nanofiber orientation changed while other structural parameters (e.g., diameter, pattern morphology) remained unchanged.

Without wishing to be bound by any theory, it is believed that MPPM formation occurs as follows. Polyester and Poloxamer 407 undergo cooperative self-assembly to form phase-separated micro-domains (FIG. 2E, I). Poloxamer 407 molecules were anchored to PLGA domains, possibly by their hydrophobic poly(propylene glycol) (PPO) segments. Most Poloxamer 407 molecules were removed during the leaching step which yields an open, permeable framework (FIG. 2E, II). A monolayer of Poloxamer 407 was left on PLGA surfaces, with the hydrophilic PEO segments pointing outward (FIG. 2E, II). The Poloxamer 407-rich domains (light shading in FIG. 2E, I, upper image) may form locally ordered mesophases (arrows, FIG. 2E, I) that guide the alignment of PLGA rich domains (dark shading in FIG. 2E, I and II, upper panels). The fact that the mesostructure feature size (e.g., cavity size, solid phase thickness) was at least 30 times larger than that reported in mesostructured silica or resin, although the same structure-directing agent was used (i.e., Poloxamer 407), shows that this structure-directing effect was long-range. This may be attributed to the fact that both polyol (i.e., Poloxamer 407) and polyester (i.e., PLGA) were nonionic. This is in contrast to conventional EISA where structure-directing is at the length scales of individual micelles or polymer chains, i.e., a shorter-range interaction. Multiple repeats of Poloxamer 407 aggregates (arrows, FIG. 2E, I) might be involved in biphasic self-assembly, somewhat analogous to microscopic phase segregation in liquid-crystalline physical gels. Such long range templating may explain the observed ECM-like feature size, which may be sufficiently large to prevent coalescence of mesostructured nanofibers due to high internal Laplace pressures. Neither covalent nor electrostatic interactions were involved, which, together with the hydrolyzable backbones of PLGA and Poloxamer 407, contributed to the biodegradability (see below) of MPPM.

FIG. 2 shows the characterization of the mesostructured membrane. FIG. 2A shows water contact angle experiments. Upper panel: side-view photographs recorded at 0 s (left), 5 s (middle) and 10 s (right) on a MPPM. Middle panel: time-lapse contact angle measurements on MPPMs. Lower panel, time-lapse water droplet volume changes calculated from contact angle measurements on MPPMs. Data are means+/−SD, n=6. FIG. 2B shows carbon 1s XPS spectra of MPPM, unleached MPPM, pure PLGA and pure Poloxamer 407. FIG. 2C is a SEM image showing the long-range concentric pattern and the domain boundary (dashed line). FFTs (right panels) were obtained from regions 1-4 in the SEM image. The arrow shows the center of the concentric domain. FIG. 2D shows a SEM image highlighting the mesostructure at one domain boundary. FIG. 2E shows proposed mechanisms: (I) the long-range structure-directing effect, and (II) the hydrophilic open framework after leaching.

### Example 3

There was great flexibility in the geometries in which the MPPM could be prepared (FIG. 3). In this example, a centimeter-scale round-ended tubular sac was formed by coating the inner surface of a glass test tube with THF solution containing PLGA and Poloxamer 407, followed by drying, leaching and membrane isolation from the test tube (FIG. 3A, I). SEM imaging of the edge of the sac (FIG. 3A, II) showed surface topography similar to that of a flat membrane (FIG. 1). To demonstrate the ability to generate hierarchical porous scaffolds, macroporous-mesostructured 3D constructs were prepared, using ˜20 micrometer SiO₂ spheres as the template (FIG. 3B) during EISA. Nanofibrous topography was preserved on the macropore surfaces. MPPMs could also be micropatterned by deposition on a silicon oxide surface micropatterned with (heptadecafluoro)-1,1,2,2-tetrahydrodecyldimethyl-chlorosilane (FIG. 3C, inset); the fluoro-silane-modified square regions repelled the polymer coating in THF solution. The boundary between nanofiber coated region and uncoated region was sharp (FIG. 3C), but nanofiber orientation did not appear to correlate with the micropattern. The MPPM could also be used as a structural support for mesh-like nanowire nanoelectronics (FIG. 3D), enabling their facile folding and rolling for potential applications in degradable and flexible electronics.

FIG. 3 shows shaping and patterning of mesostructured polymer constructs. FIG. 3A is a photograph (I) and SEM (II) of a mesostructured sac. FIG. 3B is a SEM of macroporous-mesostructured construct where the MPPM was created around a template of 20 micrometer SiO₂ microspheres. FIG. 3C shows mesostructured polymer mesh micropatterned on fluorosilane-modified rectangular domains. The boundary with the uncoated area is indicated with arrows. FIG. 3D shows a photograph of mesostructured polymer membrane used as a support for nanoelectronic devices. The dashed circle highlights one nanowire field effect transistor device. The transparent ribbons (arrows) were SU-8 structures used to support and insulate device interconnects.

### Example 4

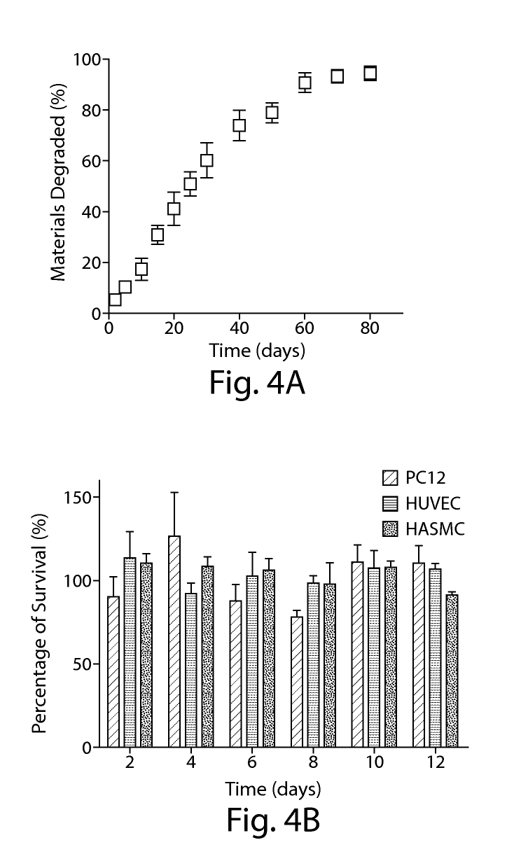

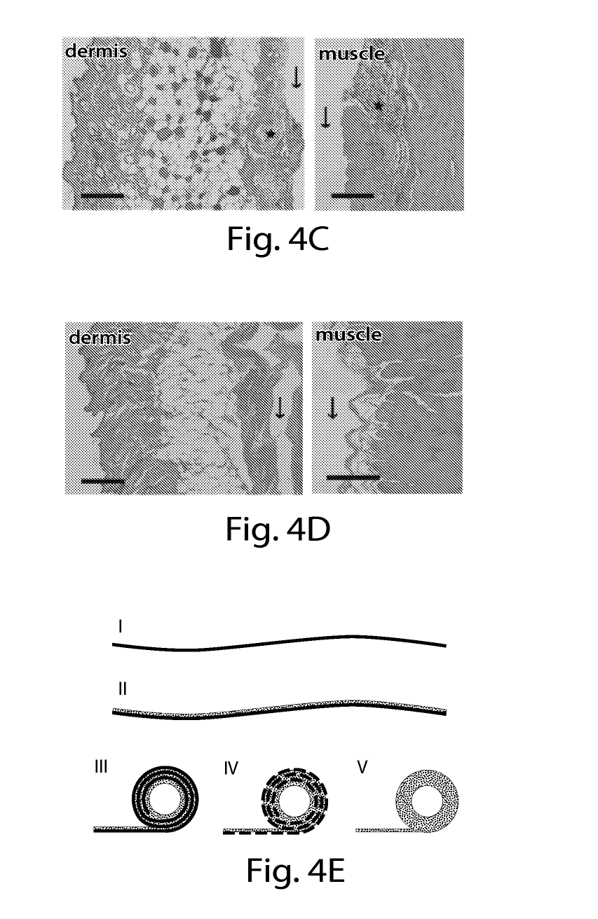

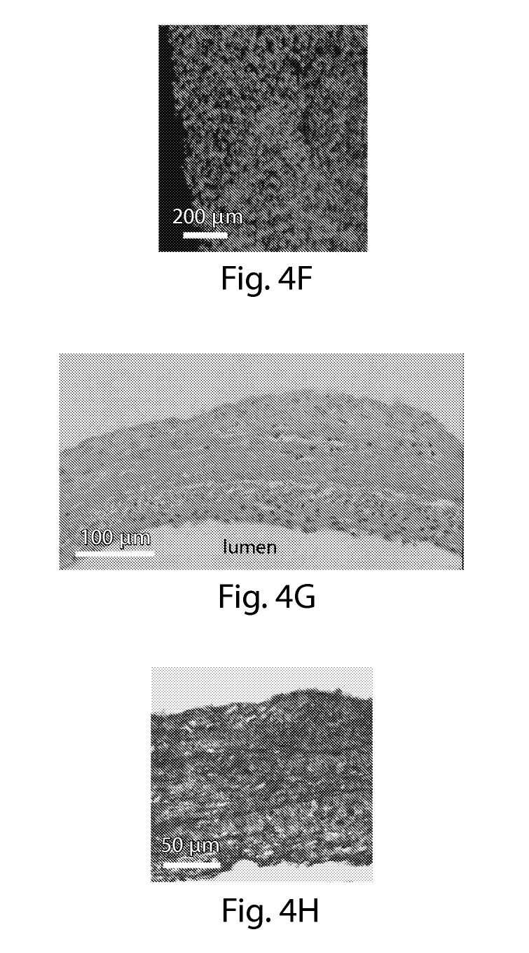

MPPM degraded in 1×PBS over a period of ˜3 months (FIG. 4A). The degradation kinetics was comparable to that previously reported for nanostructured PLGA with the same L/G ratio and similar PLGA molecular weight. The in vitro cytotoxicity of the MPPM (FIG. 4B) was evaluated in the neuroendocrine cell line PC12, human umbilical vein endothelial cells (HUVEC) and human aortic smooth muscle cells (HASMC) after MPPM modification with cyclic arginine-glycine-aspartate (cRGD) peptides (see below) to enhance cell attachment. Membranes without cRGD modifications showed poor initial cell attachment. Cell viability on RGD-modified MPPMs was similar to that in gelatin/fibronectin-coated 24-well plates over 12 days, as measured by a metabolic activity assay (MTS) (FIG. 4B). These results suggested that the MPPMs could be suitable for cell studies, such as for engineered tissue cultures.

In vivo biocompatibility of MPPM discs was assessed after subcutaneous implantation in rats for up to 1 month (FIGS. 4C and 4D, n=4 in each group). A mild inflammatory infiltrate (FIG. 4C, stars) encircled the implant cavity at day 7, consistent with results from pure PLGA implants. The inflammatory reaction decreased substantially by 1 month after implantation (FIG. 4D) when MPPMs were degraded by >70% (FIG. 4A). Sham (i.e., no MPPM) surgical procedures showed less inflammation than those with MPPMs at both time points. These results suggest that the biocompatibility of PLGA/poloxamer membranes was comparable to that of PLGA alone.

Cyclic RGD-modified MPPM (FIG. 4E, I) were used to develop engineered vascular constructs (FIGS. 4E-4H). HASMC were cultured on ˜1 micrometer thick MPPMs, with sodium ascorbate added to the media to promote deposition of natural ECM38 (FIG. 4E, II). Two days after cell seeding, the MPPM were rolled into multi-layered 3D tubular structures (FIG. 4E, III), and matured for at least 2 months to allow for thickening of the tissue layer and polymer degradation (FIG. 4E, IV and V). Cell viability on the surface of the construct was >95% (FIG. 4F). Hematoxylin and eosin and Masson's trichrome stained sections (FIGS. 4G-4H) revealed smooth muscle tissue ˜200 micrometers thick, with elongated cells and collagenous nanofibers (FIG. 4H). These results showed that the MPPMs are biodegradable and biocompatible, and suggest their potential as low-cost and versatile synthetic ECM constructs for engineered tissues.

FIG. 4 shows that the mesostructured membranes were biodegradable, biocompatible and could be used in vascular construct engineering. FIG. 4A shows gravimetric weight loss as a function of time. Membranes were weighed after rinsing and drying at each time point. Data are mean+/−SD, n=5. FIG. 4B shows cell survival by MTS cytotoxicity assay. Cultures on gelatin/fibronectin coated 24-well plate were used as controls. Data are means+/−SD, n=6. FIG. 4 C shows hematoxylin and eosin stained dermis and muscle sections immediately adjacent to mesostructured membranes 1 week after subcutaneous implantation. Scale bars: 500 micrometers. FIG. 4D shows hematoxylin and eosin stained sections 1 month after implantation. Scale bars: 500 micrometers. In FIGS. 4C and 4D, arrows mark the locations of the MPPM. FIG. 4E is a schematic of the preparation of engineered vascular constructs from MPPM. Dark=MPPM; Light=cells. FIG. 4F shows cell survival in an engineered vascular construct evaluated with a LIVE/DEAD® Viability/Cytotoxicity assay, 2 months after seeding. FIG. 4G shows hematoxylin & eosin stained sections of an engineered vascular construct, 2 months after seeding. FIG. 4H shows Masson's Trichrome stained sections of an engineered vascular construct, 2 months after seeding, highlighting the collagen matrix.

In summary, these examples show mesostructured polyol-polyester membranes that are formed by a self-assembly via an EISA process. These membranes were composed of biomaterials that have been extensively evaluated in regenerative medicine and drug delivery, were biodegradable and water permeable, and showed minimal cytotoxicity in vitro and in vivo. They may find broad applicability in a range of biomedical applications such as cell encapsulation and immunoisolation.

### Example 5

This example describes various materials and methods used in the previous examples.

Mesostructured membrane preparation: In a typical experiment, 0.2-0.33 g of poly(lactide-co-glycolide) or polylactide (PLGA or PLA, 5050 DLG 7E, 6535 DLG 7E, 7525 DLG 7E, 8515 DLG 7E and 100 DL 7E, Lakeshore Biomaterials) was dissolved in 10 mL of tetrahydrofuran (THF, Sigma). Then, 1 g of triblock poly(ethylene glycol)-poly(propylene glycol)-poly(ethylene glycol) (Poloxamer 407, Sigma) or carboxylic acid (—COOH) terminated Poloxamer 407 (used for RGD modification; see below) was added to the solution and the mixture was stirred for another 0.5-1 h. The solution was transferred onto planar or nonplanar substrates by dip- or spread-coatings, followed by solvent evaporation at ambient conditions (25° C., 30-70% relative humidity). 20 micrometers of SiO₂ microspheres were also added for the preparation of macroporous-mesostructured constructs. Annealing at 60˜70° C. on a hotplate for 10-30 min followed by cooling to ambient, and solidification of hybrid poloxamer-polyester nanocomposite membranes in humidified incubator (5% CO₂, 95% O₂, 37° C.) overnight were used to promote a stable mesostructure formation. The free portion of the poloxamer rich phase was then removed by leaching in phosphate buffered saline solution (1×PBS). Finally, the membranes were rinsed with D.I. water three times, and dried in air.

Macroporous-Mesostructured PLGA construct preparation: 100 to 1000 micrometers thick, densely packed SiO₂ spheres (20 micrometers diameter, Microspheres-Nanospheres, Cold Spring, N.Y.) were prepared by drop casting and air drying of 0.1-1 mL as-received solution on a glass slide. Then THF solutions (0.03˜0.3 mL) of PLGA (5050 DLG 7E) and Poloxamer 407 as prepared in “Mesostructured membrane preparation” were delivered into the interstitial spaces of the packed SiO₂ spheres by capillary force and were allowed to dry. After annealing and leaching, SiO₂ sphere template was removed by HF etching for 30 s.

Micropatterning of substrates for mesostructured polymer mesh preparation: In brief, silicon wafers were modified in a 1% (v/v) dichloromethane (Sigma-Aldrich Corp., St. Louis, Mo.) solution with (heptadecafluoro)-1,1,2,2-tetrahydrodecyldimethyl-chlorosilane (Gelest, Inc., Morrisville, Pa.) for 1 h, rinsed with dichloromethane and cured at 110° C. for 10 min. Following photolithographic patterning using a positive photoresist (Shipley S1805, Newton, Mass.), the fluorosilane in the exposed areas was removed by oxygen plasma (50 W for 5 minutes) and the chips were then used for dip-coating of mesostructured membranes.

Surface modification of mesostructured membranes with cyclic RGD peptides: To graft RGD to the MPPMs, Poloxamer 407 was first functionalized with —COOH terminal groups prior to EISA. In a typical synthesis, succinic anhydride (320 mg, 32 mmol, Sigma-Aldrich Corp., St. Louis, Mo.) in tetrahydrofuran (THF, 30 mL, Sigma-Aldrich Corp., St. Louis, Mo.) was added a reflux THF solution (200 mL) composed of Poloxamer 407 (5.0 g, 4 mmol, Sigma-Aldrich Corp., St. Louis, Mo.), 4-dimethylaminopyridine (DMAP, 39 mg, 3.2 mmol, Sigma-Aldrich Corp., St. Louis, Mo.) and triethylamine (323 mg, 32 mmol, Sigma-Aldrich Corp., St. Louis, Mo.). The solution was refluxed for 2 h and kept at 40° C. for 24 hours. The reaction mixture was concentrated to 20 mL and then precipitated with excessive cold anhydrous diethyl ether. The product (˜3.9 g) was collected by filtration and dried under vacuum.

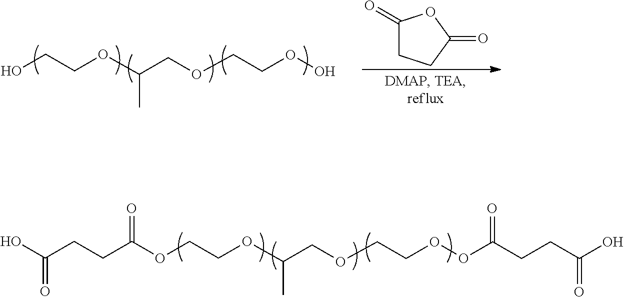

After mesostructured Poloxamer 407-COOH/PLGA membrane preparation, 30 mg of 1-ethyl-3-(3-dimethylaminopropyl) carbodiimide hydrochloride (EDC, Sigma-Aldrich Corp., St. Louis, Mo.) and 20 mg of N-hydroxysulfosuccinimide sodium (sulfo-NHS, Sigma-Aldrich Corp., St. Louis, Mo.) were dissolved separately in 1 mL of 1×PBS and then mixed together. This solution was used to cover the surface of the membrane and the reaction was allowed to occur at room temperature for 30 minutes. The membrane was then incubated with the peptide solution (1-2 mg of cyclo-(Arg-Gly-Asp-D-Phe-Lys-(PEG-PEG)) (Peptides International, Louisville, Ky.) dissolved in 1 ml of PBS) overnight at room temperature. After reaction, 100 microliters peptide reaction solution was analyzed by HPLC to measure the remaining RGD peptide in solution; the amount of RGD peptide conjugated to the film could then be back-calculated. The analyses were performed on a Hewlett Packard/Agilent series 1100 HPLC (Agilent, Santa Clara, Calif.) equipped with an analytical C18 reverse phase column (Kinetex, 75×4.6 mm, 2.6 micrometers, Phenomenex, Torrance, Calif.) with the detection wavelength at 220 nm. For Poloxamer 407-COOH/PLGA (L/G=50/50) film, the amount of RGD peptide conjugated to the film was 72.2+/−18.3 micrograms/10.0 mg film (N=5). Finally, the membrane was rinsed twice with D.I. water, air dried and UV-sterilized for one hour prior to cell culture.

Preparation of engineered vascular construct: First, ˜1 micrometer thick RGD peptide-modified PLGA membranes were prepared and sterilized. Second, human aortic smooth muscle cells (HASMC, Invitrogen) were seeded at a density of 1×10⁴ cm⁻² and cultured in Medium 231 (Invitrogen) supplemented with smooth muscle growth supplement (SMGS, Invitrogen). Sodium L-ascorbate (50 microgram/mL, Sigma) was added to the culture medium to stimulate extracellular matrix (ECM) synthesis. After two days, the cell-coated mesostructured membranes were gently lifted from the culture dish using fine forceps, rolled onto a polystyrene or glass tubular support 1.5 mm in diameter, then maintained in culture Medium 231 supplemented with SMGS and 50 microgram/mL sodium L-ascorbate for at least another 8 weeks for maturation of the vascular structure.

Hematoxylin and Eosin and Masson's Trichrome staining: The vascular constructs or rat skin tissues were cut and fixed in formalin solution (10%, neutral buffered, Sigma-Aldrich Corp.). The fixed sample was dehydrated in a series of graded ethanol baths (70% ethanol for 1 h, 95% ethanol for 1 h, absolute ethanol 3×times, 1 h each) and xylenes (2×, 1 h each), and then infiltrated with molten paraffin (HistoStar, Thermo Scientific) at 58° C. for 2 h. The infiltrated tissues were embedded into paraffin blocks and cut into 5-6 micrometer sections. Immediately prior to straining, the paraffin was removed from the sections by 2 washes with xylene, 1 min each. Then the sections were rehydrated by a 5 min wash in absolute ethanol, 2 min in 95% ethanol, 2 min in 70% ethanol and 5 min in distilled water. Standard hematoxylin and eosin staining was carried out using an automated slide stainer (Varistain Gemini ES, Thermo Scientific, Kalamazoo). Collagen secretion by HASMCs was assessed on deparaffinized sections using a Masson's trichrome staining kit (Polysciences, Inc.) according to standard protocol. Slides were examined by a blinded observer.

Viability/Cytotoxicity assays: For planar cell cultures (PC12, HUVEC, HASMC) on mesostructured membranes, cell viabilities were evaluated with an assay of a mitochondrial metabolic activity, the CellTiter 96® AQueous One Solution Cell Proliferation Assay (Promega Corp.) that uses a tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES). On days 2, 4, 6, 8, 10 and 12 of the culture, cardiac constructs were incubated with CellTiter 96® AQueous One Solution for 120 min at 37° C. The absorbance of the culture medium at 490 nm was immediately recorded with a 96-well plate reader. The quantity of formazan product (converted from tetrazole) as measured by the absorbance at 490 nm was directly proportional to cell metabolic activity in culture. Planar cultures on gelatin/fibronectin coated 24-well plate were used as controls. For each group, n=6. For vascular constructs, cell viability was evaluated using a LIVE/DEAD® Viability/Cytotoxicity Kit (Molecular Probes, Invitrogen). HASMCs were incubated with 1 micromolar calcein-AM and 2 micromolar ethidium homodimer-1 (EthD-1) for 30 min at 37° C. to label live and dead cells, respectively. Cell viability was calculated as live/(live+dead)×100.

In vivo cytotoxicity: All animals were cared for in compliance with protocols approved by the Children's Hospital Boston Committee on Animal Care, and in compliance with the NIH guidelines for the care and use of laboratory animals. To examine physiological tissue responsiveness to MPPM membranes, male Balb/c mice weighing 19-21 g were implanted subcutaneously with MPPM membranes measuring 0.5 cm×0.5 cm. Briefly, mice were anesthetized using a mixture of isoflurane and with balance oxygen dispensed through an inhalational anesthesia manifold. A 2 cm subcutaneous incision was applied in the left upper lumbar area and the MPPM membranes placed in a subcutaneous fascial pocket. The wound was closed using surgical glue. The surgical area was monitored daily for swelling, redness or for the presence of discharge. Body weight was monitored daily. Mice were euthanized at days 7, 14 and 30 (n=4 at each time point), and portions of skin and muscle overlying the implantation area were fixed in 4% formaldehyde and further processed for histological analysis.

Imaging: Scanning electron microscopy (SEM, Zeiss Ultra55/Supra55VP field-emission SEMs) was used to characterize both types of fabricated scaffold structures. Bright-field optical micrographs and epi-fluorescence images of samples were acquired on an Olympus FSX100 system using FSX-BSW software (ver. 02.02).

Statistics: Data from MPPM diameter distributions (FIG. 1E), water contact angle measurements (FIG. 2A), in vivo MPPM degradations (FIG. 4A), and in vitro cytotoxicity assessments are presented as means+/−one standard deviation. All analyses were performed using GraphPad Prism version 5.00 for Windows (GraphPad Software, San Diego, Calif.), and pb0.05 was considered statistically significant.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a porous article comprising an amphiphilic block copolymer and a hydrophobic block copolymer, the porous article comprising pores having an average pore size of between about 100 nm and about 1 micrometer, as determined using SEM.

2. The composition of claim 1, wherein the pores of the porous article has an average pore aspect ratio of at least about 2.

3. The composition of any one of claim 1 or 2, wherein the pores of the porous article has an average pore aspect ratio of at least about 3.

4. The composition of any one of claims 1-3, wherein the pores of the porous article has an average pore aspect ratio of at least about 4.

5. The composition of any one of claims 1-4, wherein the article has a mass ratio of the hydrophobic block copolymer to the amphiphilic block copolymer of between about 1:1 and about 1:10.

6. The composition of any one of claims 1-5, wherein the article has a mass ratio of the hydrophobic block copolymer to the amphiphilic block copolymer of between about 1:2 and about 1:8.

7. The composition of any one of claims 1-6, wherein the article comprises fibers.

8. The composition of claim 7, wherein at least about 80 wt % of the article comprises fibers.

9. The composition of any one of claim 7 or 8, wherein the fibers have an average diameter of between about 50 nm and about 500 nm.

10. The composition of any one of claims 7-9, wherein the fibers have an average diameter of between about 100 nm and about 200 nm.

11. The composition of any one of claims 1-10, wherein at least about 25 wt % of the article comprises the amphiphilic block copolymer and the hydrophobic block copolymer.

12. The composition of any one of claims 1-11, wherein at least about 50 wt % of the article comprises the amphiphilic block copolymer and the hydrophobic block copolymer.

13. The composition of any one of claims 1-12, wherein at least about 90 wt % of the article comprises the amphiphilic block copolymer and the hydrophobic block copolymer.

14. The composition of any one of claims 1-13, wherein at least about 95 wt % of the article comprises the amphiphilic block copolymer and the hydrophobic block copolymer.

15. The composition of any one of claims 1-14, wherein the hydrophobic block copolymer comprises a polyester.

16. The composition of claim 15, wherein at least some of the polyester comprises polylactide.

17. The composition of any one of claim 15 or 16, wherein at least some of the polyester comprises polyglycolide.

18. The composition of any one of claims 15-17, wherein at least some of the polyester comprises poly(lactide-co-glycolide).

19. The composition of claim 18, wherein the poly(lactide-co-glycolide) comprises a ratio of between about 1:100 and about 100:1 of lactide to glycolide by mass.

20. The composition of any one of claim 18 or 19, wherein the poly(lactide-co-glycolide) comprises a ratio of between about 1:30 and about 30:1 of lactide to glycolide by mass.

21. The composition of any one of claims 18-20, wherein the poly(lactide-co-glycolide) comprises a ratio of between about 1:5 and about 5:1 of lactide to glycolide by mass.

22. The composition of any one of claims 18-21, wherein the poly(lactide-co-glycolide) comprises a ratio of between about 1:2 and about 2:1 of lactide to glycolide by mass.

23. The composition of any one of claims 1-22, wherein at least some of the amphiphilic block copolymer comprises a polyol.

24. The composition of claim 23, wherein at least some of the polyol comprises poly(ethylene glycol).

25. The composition of any one of claim 23 or 24, wherein at least some of the polyol comprises poly(propylene glycol).

26. The composition of any one of claims 23-25, wherein at least some of the polyol comprises a copolymer comprising poly(ethylene glycol) and poly(propylene glycol).

27. The composition of any one of claims 23-26, wherein at least some of the polyol comprises triblock poly(ethylene glycol)-poly(propylene glycol)-poly(ethylene glycol).

28. The composition of any one of claims 1-27, wherein the average pore size is between about 200 nm and about 500 nm.

29. The composition of any one of claims 1-28, wherein the average pore size is between about 300 nm and about 400 nm.

30. The composition of any one of claims 1-29, wherein the pores are arranged as pore domains having an average dimension of between about 10 micrometers and about 1000 micrometers, as determined using SEM, the pores being substantially concentrically arranged within the pore domain.

31. The composition of claim 30, wherein the pore domains have an average dimension of between about 15 micrometers and about 500 micrometers.

32. The composition of any one of claim 30 or 31, wherein the pore domains have an average dimension of between about 20 micrometers and about 200 micrometers.

33. The composition of any one of claims 1-32, wherein at least some of the hydrophobic block copolymer is biocompatible.

34. The composition of any one of claims 1-33, wherein at least some of the hydrophobic block copolymer is biodegradable.

35. The composition of any one of claims 1-34, wherein at least some of the amphiphilic block copolymer is biocompatible.

36. The composition of any one of claims 1-35, wherein at least some of the amphiphilic block copolymer is biodegradable.

37. The composition of any one of claims 1-36, wherein the article is a membrane.

38. The composition of any one of claims 1-37, wherein the article is in physical contact with cell culture media.

39. The composition of any one of claims 1-38, wherein the article comprises mammalian cells.

40. The composition of any one of claims 1-39, wherein the article is implanted within a mammal.

41. The composition of any one of claims 1-40, wherein the article is a coating on a substrate.

42. The composition of any one of claims 1-41, wherein the article is implantable.

43. The composition of any one of claims 1-42, wherein the article further comprises a peptide.

44. The composition of claim 43, wherein the peptide comprises cyclic RGD peptide.

45. The composition of any one of claims 1-44, wherein the article has an average tensile modulus of between about 1 MPa and about 100 MPa.

46. The composition of any one of claims 1-45, wherein the article has an average tensile modulus of between about 10 MPa and about 50 MPa.

47. The composition of any one of claims 1-46, wherein the article has a contact angle of less than about 30°.

48. The composition of any one of claims 1-47, wherein the article has a contact angle of less than about 20°.

49. The composition of any one of claims 1-48, wherein the article is substantially nonionic.

50. A method, comprising:
exposing at least a portion of a substrate to a solution comprising a solvent, the solution comprising an amphiphilic block copolymer and a hydrophobic block copolymer;
removing at least some of the solvent such that the amphiphilic block copolymer and the hydrophobic block copolymer form, on the substrate, a solid comprising the amphiphilic block copolymer and the hydrophobic block copolymer; and
removing at least some of the amphiphilic block copolymer from the solid.

51. The method of claim 50, wherein the solid, after removal of at least some of the amphiphilic block copolymer, has an average pore size of between about 100 nm and about 1 micrometer, as determined using SEM.

52. The method of any one of claim 50 or 51, wherein the solid, after removal of at least some of the amphiphilic block copolymer, has an average pore aspect ratio of at least about 2, as determined using SEM.

53. The method of any one of claims 50-52, wherein the solvent is substantially immiscible in water.

54. The method of any one of claims 50-53, wherein the solvent comprises tetrahydrofuran.

55. The method of any one of claims 50-54, wherein exposing at least a portion of the substrate to the solution comprises coating at least a portion of the substrate with the solution.

56. The method of any one of claims 50-55, wherein exposing at least a portion of the substrate to the solution comprises dip-coating at least a portion of the substrate with the solution.

57. The method of any one of claims 50-56, wherein exposing at least a portion of the substrate to the solution comprises spin-coating at least a portion of the substrate with the solution.

58. The method of any one of claims 50-57, wherein removing at least some of the amphiphilic block copolymer from the solid comprises exposing the solid to an aqueous solution.

59. The method of any one of claims 50-58, wherein removing at least some of the solvent comprises exposing the coating to an environment having at least about 80% relative humidity.

60. The method of any one of claims 50-59, wherein removing at least some of the solvent comprises exposing the coating to an environment having a temperature of at least about 20° C.

61. The method of any one of claims 50-60, wherein removing at least some of the solvent comprises exposing the coating to an environment having a temperature of at least about 30° C.

62. The method of any one of claims 50-61, wherein removing at least some of the solvent comprises exposing the coating to ambient temperature and pressure.

63. The method of any one of claims 50-62, wherein the solid has a thickness on the substrate of less than about 20 micrometers.

64. The method of any one of claims 50-63, wherein the solid has a thickness on the substrate of less than about 5 micrometers.

65. The method of any one of claims 50-64, wherein the solid has a thickness on the substrate of less than about 3 micrometers.

66. The method of any one of claims 50-65, wherein the solid has a thickness on the substrate of less than about 1 micrometer.

67. The method of any one of claims 50-66, wherein the solid has a thickness on the substrate of less than about 0.5 micrometer.

68. The method of any one of claims 50-67, further comprising removing the solid from the substrate of the article as a substantially single unit.

69. The method of any one of claims 50-68, wherein the substrate is substantially planar.

70. The method of any one of claims 50-69, wherein the substrate comprises a first region having a first affinity to the solvent and a second region having a second affinity to the solvent different from the first affinity.

71. The method of claim 70, wherein the first region has a smallest dimension of less than about 1 micrometer.

72. The method of any one of claims 50-71, wherein the solution further comprises particles.

73. The method of claim 72, wherein at least some of the particles comprises TiO₂.

74. The method of any one of claim 72 or 73, wherein at least some of the particles comprises SiO₂.

75. The method of any one of claims 72-74, wherein the particles have an average dimension of between about 1 micrometers and about 100 micrometers.

76. The method of any one of claims 72-75, further comprising removing the particles after formation of the solid.

77. The method of claim 76, comprising removing the particles by exposing at least some of the particles to an etchant.

78. The method of any one of claim 76 or 77, comprising removing the particles by exposing at least some of the particles to HCl.

79. The method of any one of claims 76-78, comprising removing the particles by exposing at least some of the particles to HF.

80. The method of any one of claims 50-79, wherein at least 90 wt % of the solid comprises the amphiphilic block copolymer and the hydrophobic block copolymer.

81. The method of any one of claims 50-80, wherein at least some of the hydrophobic block copolymer comprises a polyester.

82. The method of claim 81, wherein at least some of the polyester comprises polylactide.

83. The method of any one of claim 81 or 82, wherein at least some of the polyester comprises polyglycolide.

84. The method of claim 81-83, wherein at least some of the polyester comprises poly(lactide-co-glycolide).

85. The method of claim 84, wherein the poly(lactide-co-glycolide) comprises a ratio of between about 1:6 and about 6:1 of lactide to glycolide by mass.

86. The method of any one of claim 84 or 85, wherein the poly(lactide-co-glycolide) comprises a ratio of between about 1:2 and about 2:1 of lactide to glycolide by mass.

87. The method of any one of claims 50-86, wherein at least some of the amphiphilic block copolymer comprises a polyol.

88. The method of claim 87, wherein at least some of the polyol comprises poly(ethylene glycol).

89. The method of any one of claim 87 or 88, wherein at least some of the polyol comprises poly(propylene glycol).

90. The method of any one of claims 87-89, wherein at least some of the polyol comprises a copolymer comprising poly(ethylene glycol) and poly(propylene glycol).

91. The method of any one of claims 87-90, wherein at least some of the polyol comprises triblock poly(ethylene glycol)-poly(propylene glycol)-poly(ethylene glycol).

92. The method of any one of claims 50-91, further comprising culturing mammalian cells on at least a portion of the solid.

93. The method of any one of claims 50-92, further comprising exposing at least a portion the solid to cell culture media.

94. The method of any one of claims 50-93, further comprising implanting at least a portion the solid into a subject.

95. A composition, comprising:
a porous article comprising an amphiphilic block copolymer and a hydrophobic block copolymer, the porous article having an average pore size of between about 100 nm and about 1 micrometer, as determined using SEM, wherein the porous article further comprises voids having an average dimension of between about 1 micrometer and about 100 micrometers, as determined using SEM.

96. The composition of claim 95, wherein the pores of the porous article has an average pore aspect ratio of at least about 2.

97. The composition of any one of claim 95 or 96, wherein the article has a mass ratio of the hydrophobic block copolymer to the amphiphilic block copolymer of between about 1:1 and about 1:10.

98. The composition of any one of claims 95-97, wherein the article has a mass ratio of the hydrophobic block copolymer to the amphiphilic block copolymer of between about 1:2 and about 1:8.

99. The composition of any one of claims 95-98, wherein the article comprises fibers.

100. The composition of claim 99, wherein at least about 80 wt % of the article comprises fibers.

101. The composition of any one of claim 99 or 100, wherein the fibers have an average diameter of between about 50 nm and about 500 nm.

102. The composition of any one of claims 95-101, wherein at least about 50 wt % of the article comprises the amphiphilic block copolymer and the hydrophobic block copolymer.

103. The composition of any one of claims 95-102, wherein at least some of the hydrophobic block copolymer comprises a polyester.

104. The composition of claim 103, wherein at least some of the polyester comprises polylactide.

105. The composition of any one of claim 103 or 104, wherein at least some of the polyester comprises polyglycolide.

106. The composition of any one of claims 103-105, wherein at least some of the polyester comprises poly(lactide-co-glycolide).

107. The composition of claim 106, wherein the poly(lactide-co-glycolide) comprises a ratio of between about 1:100 and about 100:1 of lactide to glycolide by mass.

108. The composition of any one of claims 95-107, wherein at least some of the amphiphilic block copolymer comprises a polyol.

109. The composition of claim 108, wherein at least some of the polyol comprises poly(ethylene glycol).

110. The composition of any one of claim 108 or 109, wherein at least some of the polyol comprises poly(propylene glycol).

111. The composition of any one of claims 108-110, wherein at least some of the polyol comprises a copolymer comprising poly(ethylene glycol) and poly(propylene glycol).

112. The composition of any one of claims 108-111, wherein at least some of the polyol comprises triblock poly(ethylene glycol)-poly(propylene glycol)-poly(ethylene glycol).

113. The composition of any one of claims 95-112, wherein the average pore size is between about 200 nm and about 500 nm.

114. The composition of any one of claims 95-113, wherein the average pore size is between about 300 nm and about 400 nm.

115. The composition of any one of claims 95-114, wherein the porous article has substantially constant porosity.

116. The composition of any one of claims 95-115, wherein the pores are arranged as pore domains having an average dimension of between about 10 micrometers and about 1000 micrometers, as determined using SEM, the pores being substantially concentrically arranged within the pore domain.

117. The composition of claim 116, wherein the pore domains have an average dimension of between about 15 micrometers and about 500 micrometers.

118. The composition of any one of claims 95-117, wherein the article is a membrane.

119. The composition of any one of claims 95-118, wherein the article is in physical contact with cell culture media.

120. The composition of any one of claims 95-119, wherein the article comprises mammalian cells.

121. The composition of any one of claims 95-120, wherein the article is implanted within a mammal.

122. The composition of any one of claims 95-121, wherein the article forms a coating on the substrate of at least a portion of the article.

123. The composition of any one of claims 95-122, wherein the article further comprises a peptide.

124. The composition of claim 123, wherein the peptide comprises cyclic RGD peptide.

125. The composition of any one of claims 95-124, wherein the article has an average tensile modulus of between about 1 MPa and about 100 MPa.

126. The composition of any one of claims 95-125, wherein the article has a contact angle of less than about 30°.

127. The composition of any one of claims 95-126, wherein the article is substantially nonionic.

128. The composition of any one of claims 95-127, wherein the article contains no more than 1% silicate.

129. A method, comprising:
inserting, into spaces between a plurality of particles, a solution comprising a solvent, wherein an amphiphilic block copolymer and a hydrophobic block copolymer are each dissolved in the solvent, and wherein the particles have an average dimension of between about 1 micrometers and about 100 micrometers; and
removing at least some of the solvent such that the amphiphilic block copolymer and the hydrophobic block copolymer form a solid comprising the amphiphilic block copolymer and the hydrophobic block copolymer.

130. The method of claim 129, wherein the particles are present on a substrate, and the solid is formed on the substrate.

131. The method of any one of claim 129 or 130, further comprising removing at least some of the particles.

132. The method of claim 131, comprising removing the particles by exposing the solid to an etchant.

133. The method of any one of claim 131 or 132, comprising removing the particles by exposing the solid to HF.

134. The method of any one of claims 131-133, comprising removing the particles by exposing the solid to HCl.

135. The method of any one of claims 129-134, wherein the particles have an average dimension of between about 10 micrometers and about 50 micrometers.

136. The method of any one of claims 129-135, wherein at least some of the particles comprise SiO₂.

137. The method of any one of claims 129-136, wherein at least some of the particles consist essentially of SiO₂.

138. The method of any one of claims 129-137, wherein at least some of the particles comprise TiO₂.

139. The method of any one of claims 129-138, comprising removing the particles by exposing at least some of the particles to an etchant.

140. The method of any one of claims 129-139, comprising removing the particles by exposing at least some of the particles to HF.

141. The method of any one of claims 129-140, further comprising removing at least some of the amphiphilic block copolymer from the solid.

142. The method of claim 141, wherein removing at least some of the amphiphilic block copolymer from the solid comprises exposing the solid to an aqueous solution.

143. The method of any one of claims 129-142, wherein the solid has an average pore size of between about 100 nm and about 1 micrometer, as determined using SEM.

144. The method of any one of claims 129-143, wherein the solid has an average pore aspect ratio of at least about 2, as determined using SEM.

145. The method of any one of claims 129-144, wherein the solvent is substantially immiscible in water.

146. The method of any one of claims 129-145, wherein the solvent comprises tetrahydrofuran.

147. The method of any one of claims 129-146, wherein at least 90 wt % of the solid comprises the amphiphilic block copolymer and the hydrophobic block copolymer.

148. The method of any one of claims 129-147, wherein removing at least some of the solvent comprises exposing the coating to an environment having at least about 80% relative humidity.

149. The method of any one of claims 129-148, wherein removing at least some of the solvent comprises exposing the coating to an environment having a temperature of at least about 30° C.

150. The method of any one of claims 129-149, wherein removing at least some of the solvent comprises exposing the coating to ambient conditions.

151. The method of any one of claims 129-150, wherein the hydrophobic block copolymer comprises a polyester.

152. The method of claim 151, wherein at least some of the polyester comprises polylactide.

153. The method of any one of claim 151 or 152, wherein at least some of the polyester comprises polyglycolide.

154. The method of any one of claims 151-153, wherein at least some of the polyester comprises poly(lactide-co-glycolide).

155. The method of claim 154, wherein the poly(lactide-co-glycolide) comprises a ratio of between about 1:5 and about 5:1 of lactide to glycolide by mass.

156. The method of any one of claim 154 or 155, wherein the poly(lactide-co-glycolide) comprises a ratio of between about 1:2 and about 2:1 of lactide to glycolide by mass.

157. The method of any one of claims 129-156, wherein at least some of the amphiphilic block copolymer comprises a polyol.

158. The method of claim 157, wherein at least some of the polyol comprises poly(ethylene glycol).

159. The method of any one of claim 157 or 158, wherein at least some of the polyol comprises poly(propylene glycol).

160. The method of any one of claims 157-159, wherein at least some of the polyol comprises a copolymer comprising poly(ethylene glycol) and poly(propylene glycol).

161. The method of any one of claims 157-160, wherein at least some of the polyol comprises triblock poly(ethylene glycol)-poly(propylene glycol)-poly(ethylene glycol).

162. The method of any one of claims 129-161, wherein the solid has a smallest cross-sectional dimension of less than about 20 micrometers.

163. The method of any one of claims 129-162, wherein the solid has a smallest cross-sectional dimension of less than about 5 micrometers.

164. The method of any one of claims 129-163, wherein the solid has a smallest cross-sectional dimension of less than about 3 micrometers.

165. The method of any one of claims 129-164, further comprising culturing mammalian cells on at least a portion of the solid.

166. The method of any one of claims 129-165, further comprising exposing at least a portion the solid to cell culture media.

167. The method of any one of claims 129-166, further comprising implanting at least a portion the solid into a subject.
